SEQUENCE LISTING (10 sequences)

SEQ ID NO: 1 - DNA, 58 bp, Artificial Sequence
  Probe
gggttccctaagggttggagtgggtaacagcctcatagtcatcacagttatagtggac

SEQ ID NO: 2 - DNA, 59 bp, Artificial Sequence
  Probe
cctcacctacactctcctatgtatttcctgcttacctctagattggatcttgctggcac

SEQ ID NO: 3 - DNA, 57 bp, Artificial Sequence
  Probe
gggttccctaagggttggacttcctggattatgggagttatgcattcaatgagtcag

SEQ ID NO: 4 - DNA, 54 bp, Artificial Sequence
  Probe
gtcatatttgccctcacgttaccattctgtgtctagattggatcttgctggcac

SEQ ID NO: 5 - DNA, 59 bp, Artificial Sequence
  Probe
gggttccctaagggttggacagctcatttcattgttgtcttcttgttctttgggccatg

SEQ ID NO: 6 - DNA, 66 bp, Artificial Sequence
  Probe
catcttcatctacatgtggccactaagcagctttctcacagactctagattggatcttgctggcac

SEQ ID NO: 7 - DNA, 50 bp, Artificial Sequence
  Probe
gggttccctaagggttggacctgctgctgcccatcctggccatgttgatg

SEQ ID NO: 8 - DNA, 54 bp, Artificial Sequence
  Probe
gcactgtgtgtgcactgccacagactgccagtctagattggatcttgctggcac

SEQ ID NO: 9 - DNA, 55 bp, Artificial Sequence
  Probe
gggttccctaagggttggactcggcttatatttcggaccacatgaaggtgcacag

SEQ ID NO: 10 - DNA, 65 bp, Artificial Sequence
  Probe
ccagggtcctcaccatgtctgtgagctctgcaacaaaggtactctagattggatcttgctggcac

(19) United States
(12) Patent Application Publication
(10) Pub. No.: US 2013/0324431 A1
(43) Pub. Date: Dec. 5, 2013

(54) OLFACTORY RECEPTOR COPY NUMBER ASSOCIATION WITH AGE AT ONSET OF ALZHEIMER'S DISEASE

(71) Applicants: Kinga Szigeti, Bellaire, TX (US); Chad A. Shaw, Houston, TX (US); Joanna Wiszniewska, Houston, TX (US)

(72) Inventors: Kinga Szigeti, Bellaire, TX (US); Chad A. Shaw, Houston, TX (US); Joanna Wiszniewska, Houston, TX (US)

(73) Assignee: Baylor College of Medicine, Houston, TX (US)

(21) Appl. No.: 13/805,544

(22) PCT Filed: Jun. 24, 2011
(86) PCT No.: PCT/US11/41858

§ 371 Date: Aug. 12, 2013

Related U.S. Application Data
(60) Provisional application No. 61/358,054, filed on Jun. 24, 2010.

(51) Int. Cl. C12Q 1/68

(52) U.S. Cl. CPC C12Q 1/6883
USPC 506/9

(57) ABSTRACT

The present invention concerns determination of risk for an early age of onset for Alzheimer's Disease in an individual. In specific embodiments, it concerns identification of copy number at chromosome 14q11.2 or a region thereof and associating a high copy number with an earlier age of onset of Alzheimer's Disease.

### TECHNICAL FIELD

The present invention generally at least concerns the fields of medicine, genetics, neurology, cell biology, and molecular biology. In particular cases, the present invention concerns the field of diagnosis and prognosis of Alzheimer Disease.

### BACKGROUND OF THE INVENTION

Alzheimer's disease (AD) is a devastating neurodegenerative disorder affecting approximately four million individuals in the US and is the most common cause of dementia in North America and Europe. (Rocca et al. 1991; Ebly et al. 1994; Kukull et al. 2002) Genetic factors play an important role in the pathogenesis of AD. Heritability is estimated between 58 and 79% based on a large population based twin study from the Swedish Twin Registry. (Gatz et al. 2006) Alzheimer Disease (AD) is the most common form of dementia and leads to progressive cognitive decline (Kukull et al., 2002). The incidence of AD rises from 2.8 per 1,000 person years in the 65-69 year age group to 56.1 per 1,000 person years in the older than 90 year age group (Kukull et al., 2002). Heritability for AD has been estimated from genetic epidemiological studies. Twin studies have shown higher concordance for monozygotic (MZ) than for dizygotic (DZ) twins: the pairwise concordance for AD was 18.6% in MZ pairs and 4.7% in DZ pairs and the corresponding probandwise concordance rates were 31.3% and 9.3% (Raiha et al., 1996).

Age at onset (AAO) of AD is an important attribute that merits therapeutic targeting. If the age of disease onset can be delayed by 5 years, it is estimated that the overall public health burden of AD will decrease by one half by 2047 (Brookmeyer et al., 1998). APOE has been found to be an important influence on AAO, and additional loci likely influence AAO of apparently sporadic AD. Genome-wide case-control and AAO association studies using SNP arrays have identified candidate regions (Bertram and Tanzi, 2008) (see the Alzgene website), however copy number variant (CNV) association studies have not yet been reported in the literature.

The observation of widespread and abundant variation in the copy number (CN) of submicroscopic DNA segments has greatly expanded our understanding of human genetic variation (Redon et al., 2006). With the advent of microarray technology allowing genome-wide ascertainment of CNVs, disease associations have been reported in schizophrenia, SLE and HIV susceptibility (Nakajima et al., 2008; Willcocks et al., 2008; Stefansson et al., 2008). CNVs influence gene expression, phenotypic variation and adaptation by altering gene dosage and genome organization (Redon et al., 2006; Stranger et al., 2007). CNVs are often multiallelic and therefore they are not adequately tagged by SNPs (Conrad and Hurles, 2007). Because of these attributes, CNVs confer a novel genetic marker map with different properties representing a supplementary approach to SNP association (McCarroll, 2008). In the present invention, there is a genome-wide CNV association with AAO of AD and the association was replicated in an independent cohort using distinct genotyping assays and analytic methods.

### BRIEF SUMMARY OF THE INVENTION

The present invention is directed to one or more systems, methods, and/or compositions that concern AD. In specific embodiments of the invention, there are methods and compositions that relate to identifying information associated with AD for an individual, particularly to copy number variation of one or more loci associated with AD.

In certain embodiments, the present invention concerns an association between age of onset (AAO) of Alzheimer's disease and copy number variation (CNV) on a region of chromosome 14 that is known to harbor a cluster of olfactory receptor genes. The identification of CNV in this region leads to a diagnostic test for predicting the age of onset of Alzheimer's disease or the probability that an individual with mild cognitive impairment (MCI) progresses to full Alzheimer's. In particular embodiments, the present invention allows one to identify younger individuals who are at risk for AD and also those for whom therapeutic options are effective.

The individual for whom the present invention is applied may or may not have or be susceptible or at risk for AD, including familial AD. The individual may or may not have risk factors for developing AD. Exemplary risk factors include age (for example, about 65 or older); family history (for example, a parent, sibling, or offspring with AD are more likely to develop AD, and the risk increases if more than one family member has the illness); and/or genetics. Risk genes may include APOE (having three common forms in apolipoprotein E-e4, apolipoprotein E-e2, and apolipoprotein E-e3).

In certain embodiments of the invention, there is a method for obtaining information about the age at onset (AAO) in Alzheimer's Disease in an individual, comprising the step of assaying copy number variation (CNV) on chromosome 14q11.2 from a sample from the individual. In particular cases, the CNV is an independent risk factor for early age at onset that may not predict AAO but predicts the increase in risk for early AAO. In specific embodiments, the copy number variation is assayed for within a genetic locus comprising one or more members of the olfactory receptor gene cluster. The copy number variation corresponds to a multiallelic cluster selected from the group consisting of OR4M1, OR4N2, OR4K2, OR4K5, and OR4K1, in certain cases. In specific embodiments of the invention, when there is a high (5+) copy number within one or more loci in chromosome 14q11.2, the individual will have an earlier AAO.

In certain embodiments, there is a method for assaying for a risk factor for early age at onset (AAO) for Alzheimer's Disease in an individual, comprising the step of assaying copy number variation (CNV) on chromosome 14q11.2 from a sample from the individual. In a specific embodiment, the copy number variation occurs within a gene locus comprising one or more members of the olfactory receptor gene cluster. In a specific embodiment, the copy number variation corresponds to one or more genes selected from the group consisting of OR4M1, OR4N2, OR4K2, OR4K5, and OR4K1. In certain cases, when there is an increase in copy number within one or more loci in chromosome 14q11.2, the individual will have a higher risk for an earlier AAO. In particular matters, when the individual will have or is at a higher risk for an earlier AAO, the individual is provided therapy for Alzheimer's Disease.

Certain embodiments of the invention encompass methods that stratify the risk of earlier AAO of AD for an individual. In specific aspects, individuals with a high copy number at chromosome 14q11.2 (including high copy number at one or more of OR4M1, OR4N2, OR4K2, OR4K5, and OR4K1) or AD patients with an early AAO are a desired group for intervention with disease modifying therapy. In certain embodiments, evaluation of olfactory receptor copy number leads to both earlier detection and, thereby, earlier administration of disease modifying therapy, in specific embodiments of the invention. Exemplary therapies for symptoms of AD include at least those that help with cognitive and/or behavioral symptoms, including cholinesterase inhibitors (for example, Donepezil, Rivastigmine, Galantamine); Memantine; tacrine; Vitamin E; antidepressants; anxiolytics; and/or antipsychotic medications, such as aripiprazole or clozapine. In some embodiments of the invention the risk factor indicates that the individual's AAO will be before age 70, 65, 60, or 55. In other embodiments of the invention, the assay may comprise multiplex ligation-dependent probe amplification. A higher risk of AAO may be assessed if the individual also has a gene that indicates high risk of AD, such as APOE4/4. A high risk may be assessed if the CNV is 2, 3, 4, 5, or greater than 5. Another embodiment of the invention is assaying for a risk factor for an earlier AAO for AD in an individual, comprising the step of assaying CNV on an olfactory receptor gene from the individual.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the present invention, reference is now made to the following descriptions taken in conjunction with the accompanying drawing, in which:

FIGS. 1A-1C summarize the discovery examples. FIG. 1A is the −log_e p values for Z-scores from the hazard function regression performed on the array data using a five-probe sliding window plotted as a function of genomic location. In FIG. 1B the −log_e p-values are plotted against the variance (surrogate for allele frequency and number of alleles). The variance filter serves to exclude spurious association from a rare CNV not highly observed in the cohort and present in only one or a few individuals at the extremes of the AAO spectrum. FIG. 1C shows the age information and CNV array data for the most significant region. AAO is represented in the red (most red at age 50) and blue (most blue at age 84) color bar to the side of each array data heatmap. Each cell in the subject age bar is adjacent to the row of the array data heatmap for that subject. Red represents younger subjects, while blue represent older subjects in a linear scale from age 55 to age 84. The blue (darker grey) and yellow (lighter grey) heatmaps convey copy number (CN) array data for the region. Each blue (dark grey)-yellow (light grey) row represents a subject and each column represents the data for a single oligonucleotide probe. Genomically adjacent oligos are shown next to each other from left to right.

FIGS. 2A-2D show quality control (QC) and genomotyping consistency between platforms and FISH confirmation. FIG. 2A shows the multiallelic Variation_{—}0316 (black bars) detected in 35% of the subjects in relation to known CNVs (orange or light grey bars). The genomic coordinates on chromosome 14 are expressed in Mb. The hashed red (mid grey) and black bars on the genomic coordinate line are olfactory receptor genes and pseudogenes, respectively. FIG. 2B shows three subjects ascertained on both the aCGH and SNP arrays, having diploid, one copy gain and two-copy gain genotypes in the three respective panels. The aCGH data is plotted by log_{—}2-ratio and the SNP array data is visualized as copy number state after segmentation with the HMM algorithm. Multiplex ligation-dependent probe amplification (MLPA) assays are shown in FIG. 2C for three HapMap samples harboring the diploid, one-copy gain and two-copy gain genotypes (array data is equivalent of panel B). Confirmation of gene dosage and genomic location by FISH using G248P88752A11 fosmid clone (green signal) and RP11-52401 control BAC clone (red) are demonstrated in FIG. 2D.

FIGS. 3A-3D show olfactory receptor cluster CN association with AAO of AD in the replication cohort. FIG. 3A boxplots depict the dosage association of the inferred CNV Variation_{—}0316 with AAO found online using the Database of Genomic Varients hosted by The Centre for Applied Genomics. There is an association of increased CN state with earlier AAO, the largest association signal emerging from the CN state 5+. FIG. 3B shows the corresponding survivorship curves for each of the groups depicted in the boxplots, the lowest curve represents CN 5+, the next higher curve represents CN 4, the next curve represents CN 2, and the highest curve is CN of 3. Subsequently, Cox proportional hazard regression was performed using the inferred CNV Variation_{—}0316 incorporating APOE and gender into the model. FIG. 3C boxplots show the dosage association with AAO this time separating against the various APOE backgrounds. In each APOE class increased CN state is associated with earlier AAO, again the largest signal emerging form the CN state 5+. The time to event curves in FIG. 3D contrast the CN2 and CN3 states (red, top most two curves) with the CN5+ states (green, bottom most two curves) on the APOE N/4 (light) and APOE 4/4 (dark) backgrounds.

FIG. 4 shows calculating of D′ by applying the 4-gamete rule from the SNP dataset for the region encompassing the multiallelic CNV on chromosome 14q11.2 (reference sequence position 19.3-20 Mb). The bars above the LD map depict the location of the MLPA assay and the FISH probe. The CNV locus (DGV 8765) is depicted as a black bar, the region of gene dosage association is red (mid-grey) and rs11849055 associated with AAO in the recessive model is depicted as Blue Square (light grey). This SNP is not in LD with the CNV, thus may represent mutational load at the locus.

FIG. 5 shows the 5+ CN state array data visualized for the 12 subjects identified by Genome-Wide Human SNP Array 6.0 (Affymetrix). The region of dosage association is highlighted by the bar labeled Dosage association. The coverage of the array diminishes centromeric from the region of interest.

FIG. 6 shows the normalized data (mean zero, variance one) for both the Affymetrix and MLPA assays. The curves depict a probability density estimate of the data, while the hash marks below depict the actual data points colored according to the final CN state calls. The CN state calls for the Affymetrix and MLPA data were determined separately each using a 4 component Gaussian mixture model. The initial model parameterizations were determined using the Partitioning About Medoids (PAM) method as provided in the R cluster analysis package using 4 medoid components. Subsequently, the means, variances and frequencies of the cluster classes determined were used as a prior to call CN states in each cohort. Classes were assigned based on maximum posterior density of each individual value based on the 4 component mixture parameterized by the PAM result. The colored hash marks beneath each density curve represent the state calls, with red (far left cluster at −1) indicating the lowest CN state and dark green (cluster at 1) the 5+ state. The vertical lines represent the mean value of the data assigned to each CN class by the mixture model.

FIGS. 7A-7B show that Eigenstrat principal component analysis was performed on the AD subjects with genome wide SNP and CNV data ascertained on the Genome-Wide Human SNP Array 6.0 (Affymetrix). These subjects were enrolled consecutively in the TARC cohort without selection bias and represented all CN states in comparable proportions to the metaanalysis. All subjects were self-reported as Caucasian. FIG. 7A and FIG. 7B depict the lack of clustering of the various copy number states using the first two principal components for the SNP and CNV datasets, respectively. The CNV PCA confirms the lack of spurious association caused by systemic effect on the individual CN states, and the SNP PCA indicates that the CN states are not a result of population substructure and admixture.

### DETAILED DESCRIPTION OF THE INVENTION

In keeping with long-standing patent law convention, the words “a” and “an” when used in the present specification in concert with the word comprising, including the claims, denote “one or more.” Some embodiments of the invention may consist of or consist essentially of one or more elements, method steps, and/or methods of the invention. It is contemplated that any method or composition described herein can be implemented with respect to any other method or composition described herein.

As used herein, an “individual” is an appropriate individual for the method of the present invention. Individuals may also be referred to as “patients,” or “subjects.”

The term “age of onset” or “AAO” is used herein to refer to age at which an individual displays symptoms of AD or is diagnosed with AD. Diagnosis of Alzheimer's disease may be made by subjective or objective means known to one of skill in the art. Average AAO may be determined for individual studies, but in one embodiment the average AAO is 72.5 years old. An earlier AAO is earlier that the average AAO and a later AAO is an AAO at an older age than the average.

The term “therapy” or “treatment” refers to a process that is intended to produce a beneficial change in the condition of the patient. A beneficial change can, for example, include one or more of the following: restoration of function; reduction of symptoms; limitation or retardation of progress of a disease, disorder or condition; or prevention, limitation or retardation of a deterioration of a patient's condition, including cognitive function. Such therapy can involve, for example, administration of an Alzheimer's therapy.

The term “essentially equal” or “about” as used herein, refers to equal values or values within the standard of error for such values.

As used herein, “risk” refers to a predictive process in which the probability of a particular outcome is assessed. In specific embodiments the risk is the probably that an individual develops AD at an earlier age than the average AAO.

The term “probable” or “probability” as used herein, has the normal English meaning, including the likelihood or chance that something is the case. A calculated probability may correspond to the mathematical meaning, and obey the mathematical laws of probability or may also be weighted or labeled to ease computational costs at the possible expense of accuracy. For example, a probable AAO may be said to be earlier, average or later, or may be represented by a number, such as around 10 years earlier, 5 years earlier, average, 5 years later, 10 years later or the like. In embodiments of the invention, the probable AAO is calculated from the olfactory CNV as described herein. The probable AAO may also be calculated from a combination of the olfactory CNV and other factors that affect AAO or risk of AD, such as APOE4/4.

AD is the most common form of dementia and leads to unrelenting cognitive decline (Kukull et al. 2002). The incidence of AD rises from 2.8 per 1,000 person years in the 65-69 year age group to 56.1 per 1,000 person years in the older than 90 year age group (Kukull et al. 2002). With increased longevity the prevalence of AD in the elderly represents a major public health problem (Rice et al. 1993). Definite AD is a pathological diagnosis and is characterized by accumulation of the amyloid beta-peptide in amyloid plaques and neurofibrillary tangles (Joachim et al. 1988). Clinical diagnosis of probable (PAD) and possible AD can be established by the NINCDS-ADRDA criteria (McKhann et al. 1984). Large autopsy series have shown that the ante mortem diagnosis of AD via clinical criteria is correct in approximately 80-90% of patients (Joachim et al. 1988).

AAO of AD is an important phenotype and likely to be highly relevant clinically for disease modifying therapies. Although estimating disease onset (by single question or standardized, validated structured interview with landmark event to facilitate recall(Doody et al. 2004)) retrospectively likely introduces noise due to the inaccuracy of the estimate, it has proven adequate in detecting the effect of APOE4 on AAO even by the single question method. The structured method has significant (p<0.005 by Kendall coefficient) interrater concordance. As in this late onset, disease intervention that delays onset of symptoms by 5 years would reduce the public health burden of the disease by half by the year 2048 (Brookmeyer et al. 1998)

AD Genetics

The heritability is estimated between 58 and 79% based on twin studies, including a large population based twin study from the Swedish Twin Registry (Gatz et al. 2006). Rare mendelian forms of AD have confirmed and elucidated pathways involved in amyloid accumulation, but are known to only contribute to a small percentage of AD (Kukull et al. 2002). Several genes have been associated with AD, of which APOE is the strongest risk factor representing an odds ratio between 2.6-14.9 in Caucasians. Other association studies encountered odds ratios of less than 3 or more than 0.5 (Alzgene website). Despite the relatively small attributable risk of these previously identified loci, their identification has stimulated research and the development of therapeutic strategies that may be helpful for all patients with AD.

Copy Number Variation

With the advent of whole-genome scanning methods that enable interrogation of the humane genome at a resolution between that of cytogenetic analysis and DNA sequencing, a new perspective on human genetic variation was observed; the widespread variation in the copy number of submicroscopic DNA segments. CNV is defined as a DNA segment that is 1 kb or larger and is present at variable copy number in comparison with the reference genome (Feuk et al. 2006). CNVs are a group of structural variants and can be classified as deletions, duplications, deletions and duplications at the same locus, multi-allelic loci, and complex rearrangements. Studies with CNVs encounter multiple challenges in this early phase. The terminology is under development and is used in both normal and disease context at the present time (McCarroll and Altshuler 2007).

CNVs are major contributors to genetic variance, thus, it is conceivable that they may confer susceptibility to or cause disease (Redon et al. 2006). CNVs influence gene expression, phenotypic variation and adaptation by altering gene dosage (Redon et al. 2006). A recent study of gene expression variation as a model of complex phenotype found that 18% of the gene expression traits were associated with CNVs (Stranger et al. 2007).

CNVs have been identified in Mendelian disease and were found to be associated with complex traits. Duplication of APP causes autosomal dominant early-onset AD with cerebral amyloid angiopathy (Rovelet-Lecrux et al. 2006), duplication and triplication of SNCA causes familial Parkinson disease (Singleton et al. 2003), and LMNB1 duplication causes leukodystrophy (Padiath et al. 2006), all confirmed by segregation of the disease phenotype with the CNV in autosomal dominant families. CNVs were found to be associated with disease phenotypes, including FCGR3B copy number variation with susceptibility to systemic autoimmunity (Fanciulli et al. 2007), low copy number and high copy number of complement component C4 with susceptibility and protection from systemic lupus erythematosus, respectively (Yang et al. 2007), increased copy number of CCL3L1 with markedly enhanced HIV susceptibility (Gonzalez et al. 2005) and GSK3B gene copy number variation with bipolar disorder (Lachman et al. 2007).

The recombination events resulting in CNVs may be frequent. The earliest estimates for the frequency of recombination events leading to CNV comes from studies on CMT1A duplication and it occurs de novo in 47-90% of sporadic cases (Nelis et al. 1996) (Hoogendijk et al. 1992). Recently it has been shown at the whole genome level, that about 0.3% of biallelic CNV genotypes exhibit mendelian discordance in parent-offspring trios (Redon et al. 2006), several fold higher than SNPs.

Databases to catalogue these structural variants have been created, the two main ones are the Toronto Database of Genomic Variants and the Human Structural Variation Database. The Toronto Database of Genomic Variants contains 17641 CNV entries at 5672 loci at present. This covers approximately 500 Mb (18.8% of the euchromatic genome) (Scherer et al. 2007). These loci were captured in samples from usually young, healthy individuals, thus the relevance as normal for a common disease affecting the elderly, such as AD, is not clear.

Pathogenic CNVs may be more amenable to therapy that other types of genetic variation. CNVs alter gene dosage thus modification by small molecules may be possible in contrast to mutation events resulting in loss of function or toxic gain of function. The CMT1A duplication rat model treated with a progesterone antagonist had correction of gene dosage and clinical and pathological improvement (Sereda et al. 2003).

Human Olfactory System

The human olfactory system involves the olfactory neuroepithelium located in the superior turbinate, the dorsal area of the nasal vault and in the superior part of the nasal septum. The olfactory sensory neurons (OSN) are bipolar cells with dendrites that end in a knob from which 10-25 cilia project. The cilia contain the G-protein coupled receptors (OR receptors) that bind the odour molecules. Only one olfactory receptor gene from one allele is expressed in any olfactory sensory neuron by allelic exclusion. The ORs are highly variable in copy number. The OSNs are the first order neurons; their axons penetrate the cribriform plate and synapse in the glomeruli of the olfactory bulb with the mitral and tufted cells (second-order neurons). The axons of the mitral and tufted cells project to the olfactory tract, the anterior olfactory nucleus, the pyriform lobe (including the entorhinal cortex) and to the amygdale and hippocampus of the limbic system. The entorhinal cortex is the secondary olfactory cortex and interestingly the presumed first stage of AD pathology in the model by Braak and Braak.

Olfactory deficit has been studied in aging, cognitive impairment, MCI and AD. Clinical observations suggest that approximately 90% of patients with early-stage AD exhibit olfactory dysfunction, as measured by psychophysical and electrophysiological tests. Prospective cohort studies established the risk olfactory deficit infers for the development of cognitive impairment. A prospective study of 1920 subjects found a significant association between olfactory impairment at baseline and 5-year incidence of cognitive impairment (odds ratio (OR)=6.62, 95% confidence interval (CI)=4.36-10.05) (Schubert et al. 2008). Another study of 1,604 nondemented older adults, women with anosmia who possessed at least 1 apolipoprotein E4 allele had an odds ratio of 9.71 for development of cognitive decline over the ensuing 2 years, compared with an odds ratio of 1.90 for women with no olfactory dysfunction and at least 1 such allele (Graves et al. 1999). Olfactory deficit in subjects with MCI predicted conversion to AD at two year follow up (Devanand et al. 2000). A recent study compared olfactory sensitivity, identification and discrimination in patients with MCI and AD (Mesholam et al. 1998). MCI patients were impaired in olfactory sensitivity and identification, while the AD patients were impaired in all three domains. Odor discrimination and identification performance correlated more prominently than detection thresholds with performance on multiple neuropsychological tests (Djordjevic et al. 2008). Congruent with the early olfactory loss of AD is the early pathological involvement of the olfactory bulb and anterior olfactory nucleus. Neuropathological series found marked cell loss and the presence of disease-related pathology: neuritic plaques and neurofibrillary tangles (Ohm and Braak 1987).

The olfactory dysfunction associated with cognitive decline or AD does not have to be etiologic. The anatomical connection to the medial temporal lobe and the observed AD pathology in the olfactory bulb and anterior olfactory nucleus may be an early manifestation of the AD process. This interpretation suggests that the olfactory dysfunction may be an early clinical sign of the AD pathology affecting the brain.

Although the “olfactory hypothesis” suggesting an etiologic role of the olfactory pathway is currently not considered the main mechanism for AD, data presented within the Examples warrants brief consideration of the hypothesis. There are two current hypotheses: “olfactory vector” and “olfactory damage”. However, the embodiments of the invention are not bound by either of these hypotheses. The olfactory vector hypothesis suggests that xenobiotics (viruses, toxic agents, heavy metals and air pollution) enter the brain through the olfactory epithelium. The anatomy of the nose is well suited for the transfer of exogenous agents into the brain. The olfactory nerve (cranial nerve I) is uniquely vulnerable to penetration of xenobiotics. Unlike other receptor cells, these cells are also first-order neurons, projecting axons directly to the brain without an intervening synapse and unlike other first-order neurons receive little benefit from the protection of the blood-brain barrier or the blood-nerve barrier. Once internalized into the olfactory bulb, some xenobiotics penetrate into higher brain regions, often along neurotransmitter-specific lines.

The olfactory damage hypothesis suggests that damage to the afferent olfactory pathways may predispose genetically or otherwise susceptible individuals to AD, regardless of the cause of the olfactory damage. Major nongenetic risk factors for AD, such as advanced age and head trauma are directly related to olfactory system damage. The smell loss associated with advanced age is likely secondary to the occlusion of the foramina of the cribriform plate by appositional bone growth and to cumulative damage to the olfactory neuroepithelium from bacteria, viruses, and other xenobiotic agents. Further supporting the “olfactory damage” hypothesis for AD is the finding that removal of the olfactory bulbs of both rats and mice leads to decreased performance on cognitive tasks not dependent on olfaction. Rather, these tasks depend on the degenerative disruption of interconnections with higher brain regions, such as the connection between the olfactory and septohippocampal systems (Kurtz et al. 1989).

Olfactory Assessment

Quantitative measures of olfactory dysfunction include tests that assess detection threshold, identification, discrimination and memory. Odor identification has been studied in AD and MCI and appeared to be the most sensitive method to measure the olfactory dysfunction. The odor identification test evaluates the subject's ability to identify an odorant at the suprathreshold level. The multiple choice identification test is the most sensitive and specific procedure to assess identification. In this test the subject identifies the stimulus from a list of odors. The most widely used measure is the University of Pennsylvania Smell Identification Test (UPSIT). The UPSIT is a multiple-forced-choice odor identification test. For each odorant there are four possible responses and the subject is required to choose one even if smell is not perceived. It requires 10-15 minutes to administer. The UPSIT consists of 40 odorants in 4 booklets, 10 odorant in each booklet. The odorant is located on a brown strip in microencapsulated crystals at suprathreshold level. The strip has to be scratched with a pencil and then one of the 4 choices marked. The measure has been validated for short-term, long-term and test-retest reliability. (Doty et al. 1989) Normative data for the UPSIT include a score on the 1-40 scale and percentile ranks for men and women across the entire age span.

Methods to Detect CNVs and Define Alleles

Two major high-resolution methods are available currently for detection of gene dosage at the genome level (Carter 2007) (Scherer et al. 2007), in addition to deep sequencing methodologies. Copy number state can be ascertained by aCGH or derived from SNP arrays (Table 1). One major difference between the aCGH and the SNP derived information is the lack and thereof of an amplification step, respectively, which reduces the resolution of the latter. Another important difference is the derivation of CNV state in relation to a reference genome: while aCGH uses a single genome in every experiment as a common denominator (1 to 1 comparison), the SNP arrays use a bioinformatically generated reference genome from multiple cases (1 to average comparison). In aCGH the labeling is controlled at every single array, while in a SNP array the reference value will depend on the normalization efficiency and the allele frequency of any given CNV. The accuracy and sensitivity for the detection of CNV has recently been studied (Cooper et al. 2008) which suggests that aCGH has a superior dynamic range and sensitivity/specificity. In addition, array CGH has the flexibility of custom design. A database of experimentally and bioinformatically tested probes is available in eArray. Any of these methodologies may be used for the detection of CNVs in embodiments of the invention.

TABLE 1
Comparison of aCGH and SNP array for
inferring copy number variation.

| | aCGH | SNP Array |
|---|---|---|
| Design | Main application | Secondary application |
| Probes empirically tested | Yes | No |
| Amplification step | No | Yes |
| Reference sample | Intraexperimental | Extraexperimental, |
| | | reference mean of >40 |
| | | samples |
| Interarray variability | Compensated for by | Compensated for by |
| | reference sample | normalization |
| Intraarray variability | Compensated for by | Compensated for by |
| | normalization | normalization |
| Optimization for | For CNV | For SNP calling |
| sensitivity and specificity | | |

Age at Onset Analysis

AAO is highly variable in AD, and represents a clinically important endophenotye. The potential utility of AAO in identifying genetic factors in AD as a proof of principle was demonstrated by Macgregor et al (Macgregor et al. 2006). One possibility is to use a time-to-onset analysis using hazard function regression and treating the array values (normalized numeric data or CNV calls) as informative covariates. Previous authors have used AAO analysis to investigate complex disease (Li and Fan 2000), but this disclosure is the first to use DNA copy number data for this kind of analysis. Fortunately, the statistical methods for hazard function regression are well established and little new is required. Data may be analyzed with both parametric and non-parametric hazard function analysis. An added benefit of the hazard regression approach is the flexibility of the models to include possible population structure information. In one scenario, an independent population structure variable can be included as an additional covariate. Fitting such a model provides a simple but explicit method to determine and to account for possible stratification effects.

Sequential Addition in Association Analysis

Control individuals by definition do not have an age of disease onset. MacGregor et al (Macgregor et al. 2006) developed a sequential addition procedure to test genotypic differences between cases and controls in the case-only quantitative trait situation, which would include Age of onset. The sequential addition procedure builds on the ordered subset analysis described by Hauser and was used in linkage analysis. According to this method, if the quantitative trait is important in the genotype-phenotype relationship, then one end of the distribution of trait values will contribute disproportionately to the association signal and offset the penalty for multiple testing. The procedure operates by building a collection of nested sets of cases which are ordered by age at onset. These nested sets are sequentially tested from one extreme towards the other against the control CNV data. Significance is established by a permutation procedure which directly accounts for multiple testing. The ordered subset analysis identified distinct signals from the analysis using regression analysis of AAO as a quantitative trait in the same dataset (Scott et al. 2003). Analysis of age at onset as a QTL identifies loci that explain variation in age at onset across the entire AAO distribution. In contrast, ordered-subset analysis identifies loci that have stronger effects in subsets of patients defined by a continuous covariate. Although the two methods have common goals (identifying genes affecting AAO of AD), the approaches likely detect loci with different mechanisms of action. Either or both methods may be used (HFR and SA) to comprehensively interrogate the dataset, in embodiments of the invention.

AD is one of the most significant public health problems and likely to increase in its burden to society, considering projected increases in longevity and number of aging individuals (Kukull et al., 2002). Delay of AAO of AD could result in marked decrease in prevalence and therefore AAO is an important attribute of disease and a desired therapeutic target. Although AAO can only be estimated, the usefulness of determining symptom onset via the single question inquiry method has been demonstrated by the identification of the influence of the APOE4 allele on AAO (Corder et al., 1993). Estimation of AAO or estimation of the risk of early AAO could increase detection and initiate early therapy of AD in patients.

Recently copy number variants (CNVs) have been recognized as important mechanisms of genetic variation contributing to disease phenotypes. Alzheimer disease (AD) exhibits high heritability of both the occurrence of disease and age at onset (AAO). The inventors pursued a genome wide approach to identify loci that modify AAO of AD using CNVs as a genetic marker map. The inventors performed a cases-only CNV genome-wide association studies (GWAS) with AAO of AD in the discovery cohort (N=40) consisting of APOE4 non-carriers to increase power by eliminating the variance of AAO attributable to APOE4. Subsequently the inventors performed a replication study in a cohort of 507 subjects comprised of patients with all APOE genotypes using independent experimental and statistical methodology which also incorporated the APOE genotype information. A chromosomal segment was identified on 14q11.2 (reference sequence position 19.3-19.5 Mb) where gene dosage is associated with AAO of AD (genome-wide adjusted p<0.032) in the discovery study. Interestingly, this region encompasses a cluster of olfactory receptors. The replication study confirmed the dosage association (p=0.035) for the olfactory receptor locus independent of and in addition to the association of APOE. The association was present on all APOE backgrounds and the multi-copy gain (5+) conferred the highest risk for early AAO (Odds Ratio for AAO<72 was 5.8 (CI 1.7-20) p=0.001). Thus, in specific embodiments of the invention, this olfactory receptor region comprises a modifier of AAO of AD. This cases-only copy number variation genome wide association study with AAO of AD and have detected association of gene dosage of the olfactory receptor region on chromosome 14q11.2 with a substantial association (9.75 years difference in median AAO of AD). Details are found in the examples.

### Kits of the Invention

Any of the compositions described herein may be comprised in a kit. In a non-limiting example, a device or reagent for assessing copy number of a specific locus at chromosome 14q11.2 may be comprised in a kit. The kits will thus comprise, in suitable container means and suitably aliquoted, a device or reagent of the present invention. In a specific embodiment, the kit comprises a microchip that comprises nucleic acids that are part of or are complementary to one or more regions at chromosome 14q11.2. In specific embodiments, the microchip may comprise oligonucleotides that are complementary to a strand of genomic DNA within the region of chromosome 14q11.2, including reference sequence position 19.3-19.5 Mb, and may include at least part of one or more of an olfactory receptor gene, including, for example, one or more of OR4M1, OR4N2, OR4K2, OR4K5, or OR4K1. In some embodiments, primers or probes are utilized that recognize one or more of OR4M1, OR4N2, OR4K2, OR4K5, or OR4K1.

The components of the kits may be packaged either in aqueous media or in lyophilized form. The container means of the kits may generally include at least one vial, test tube, flask, bottle, syringe or other container means, into which a component may be placed, and preferably, suitably aliquoted. Where there are more than one component in the kit, the kit also will generally contain a second, third or other additional container into which the additional components may be separately placed. However, various combinations of components may be comprised in a vial. The kits of the present invention also will typically include a means for containing the reagent containers in close confinement for commercial sale. Such containers may include injection or blow molded plastic containers into which the desired vials are retained.

### EXAMPLES

The following examples are included to demonstrate preferred embodiments of the invention. It should be appreciated by those of skill in the art that the techniques disclosed in the examples that follow represent techniques discovered by the inventors to function well in the practice of the invention, and thus can be considered to constitute preferred modes for its practice. However, those of skill in the art should, in light of the present disclosure, appreciate that many changes can be made in the specific embodiments that are disclosed and still obtain a like or similar result without departing from the spirit and scope of the invention.

### Example 1

### Olfactory Copy Number Association with Age of Onset of Alzheimer's Disease

A cases-only genome-wide CNV association study was performed looking for loci affecting AAO of AD. In the discovery cohort data was collected on array comparative genome hybridization and binned probe level array data was the predictor in a hazard function regression with AAO as the outcome. A correction for multiple testing in the genome wide analysis was performed via a simulation study performing 1000 permutations of the patient labels. The replication study was performed on SNP array and inferred CNVs were the predictors in a hazard function regression. The gene dosage and genomic location was confirmed by FISH for the most common allele using HapMap cell lines. A chromosomal segment on 14q11.2 (reference sequence position 19.3-19.5 Mb) was identified where gene dosage is associated with AAO of AD (genome-wide adjusted p<0.031). Interestingly, this region encompasses a cluster of olfactory receptors. The association of the 14q11.2 olfactory receptor gene cluster CNVs with AAO of AD was confirmed in an independent cohort by independent experimental and statistical methodology and again demonstrated a clear gene dosage effect (p=0.00099). Gain in this region (N>2 copy number) was associated with earlier AAO, and loss was associated with later AAO. Olfactory receptor CNV on chromosome 14q11.2 is associated with AAO of AD. This observation indicates that olfactory dysfunction in AD is directly involved in the pathological process leading to disease by implicating this olfactory receptor region as a modifier of AAO, in certain embodiments of the invention.

### Example 2

### Exemplary Subjects and Methods

### Subject Cohorts

The discovery and replication cohorts included 40 and 507 subjects with Probable AD by NINCDS-ADRDA criteria (McKhann et al., 1994), respectively. The discovery cohort samples and associated phenotypic data were collected at the Alzheimer Disease and Memory Disorders Center of Baylor College of Medicine (Doody et al., 2005). The methodology of the Texas Alzheimer Research Consortium project has been described in detail elsewhere (Waring et al., 2008). These institutions participated in the collection of samples and phenotypic data from the replication cohort following a standardized IRB-approved study protocol. The discovery cohort was ascertained as the first 40 consecutive APOE non-carriers with the permission of one APOE 4 allele if the subject had early AAO, as studies consistently reported that one APOE4 allele has minor effect on AAO compared to homozygosity for APOE4. This design removes the variance of AAO originating from APOE thus increases power. Further description of the cohorts is available below.

### AAO Phenotyping

AAO was determined with two standardized methods in both cohorts: i) caregiver estimate by prompted standard question regarding onset of symptoms and ii) physician estimate of duration of illness using a standardized and validated structured interview with landmark event to facilitate recall (Doody et al., 2004).

### Detection of Copy Number Variation and Test of Association in the Discovery and Replication Cohorts

The inventors used independent strategies in the evaluation of CNV association with AAO of AD in each of the respective cohorts. The discovery data were generated by array comparative genome hybridization (aCGH). A time-to-event parametric hazard function regression was performed to analyze the association between copy number state (explanatory variable, as measured by microarray log 2-ratios) and AAO (the regression outcome variable).

In the replication cohort two genotyping assays were used (see below) to determine copy number state. Approximately half of the cohort (N=243) was assayed on Affymetrix array. The log 2ratios were generated in the Genotyping Console 3.0.1 software after regional GC correction using 78 concomitantly ascertained normal controls as reference. A Gaussian mixture model applied to the normalized mean intensities was used to analyze CN state (see below). The CN calls were validated by MLPA in a subset of subjects and in all subjects with the 5+ CN state. Subsequently the high throughput MLPA assay was used to genotype an additional 264 samples in the replication cohort. CN calls for the MLPA assay were also made by use of a Gaussian mixture model (see below). The distribution of CN states within the assays is depicted in Table 2. Cox proportional hazard analysis was performed using the inferred CNV Variation_{—}0316 (see the Database of Genomic Variants website) dosage incorporating gender and APOE status (number of APOE alleles as categorical variable) in the proportional hazard model. The analysis used the Efron method to handle ties, and the implementation of the regression was provided by the R survival analysis package.

TABLE 2
Summary of AAO distribution for AD cohort genotyped
with two methods (Affymetrix and MLPA)

| | Affymetrix | | | MLPA | | |
|---|---|---|---|---|---|---|
| CN | N | mean ± SD | range | N | mean ± SD | range |
| 2 | 102 | 72.1 ± 8.7 | 51-98 | 117 | 71.3 ± 9.3 | 48-90 |
| 3 | 89 | 71.8 ± 8.8 | 47-89 | 95 | 72.4 ± 8.8 | 48.5-89 |
| 4 | 40 | 70.4 ± 9.8 | 52-90 | 44 | 69.8 ± 8.5 | 49-90 |
| 5+ | 12 | 65.3 ± 6.6 | 53-77 | 8 | 64.9 ± 10.8 | 54-83 |
| Total | 243 | 71.4 ± 8.9 | 47-98 | 264 | 71.3 ± 9.1 | 48-90 |

### Example 3

### Olfactory Receptor Cluster Copy Number Association with AAO and Ad in the Discovery Cohort

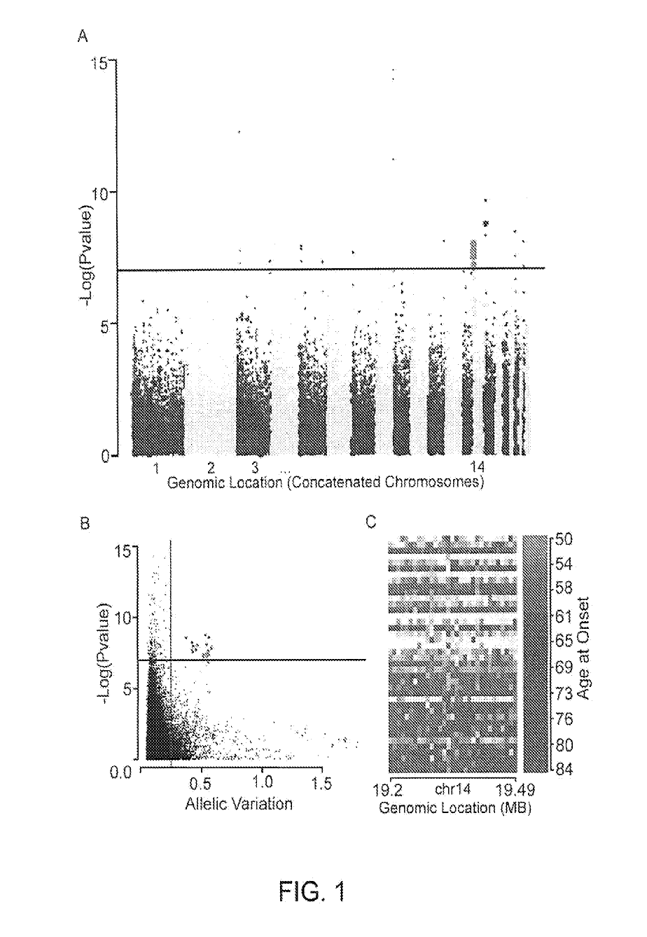

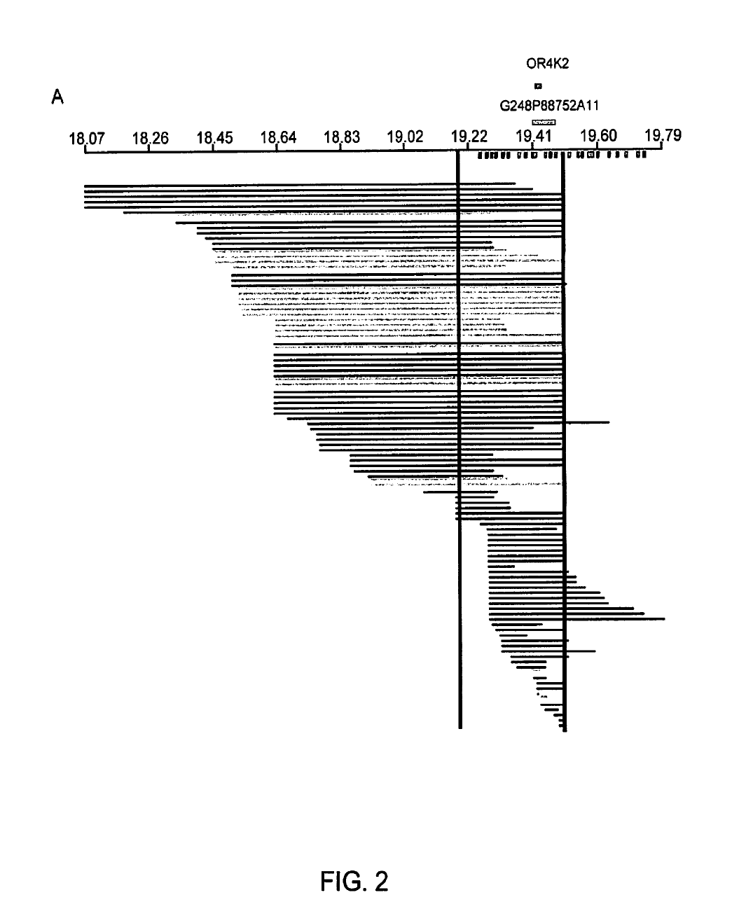

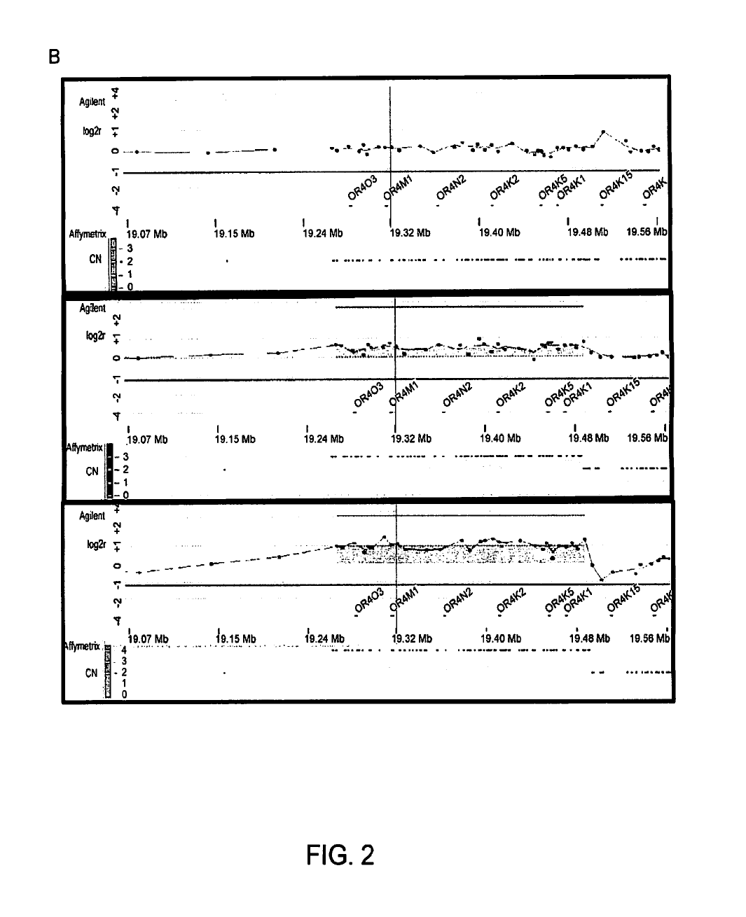

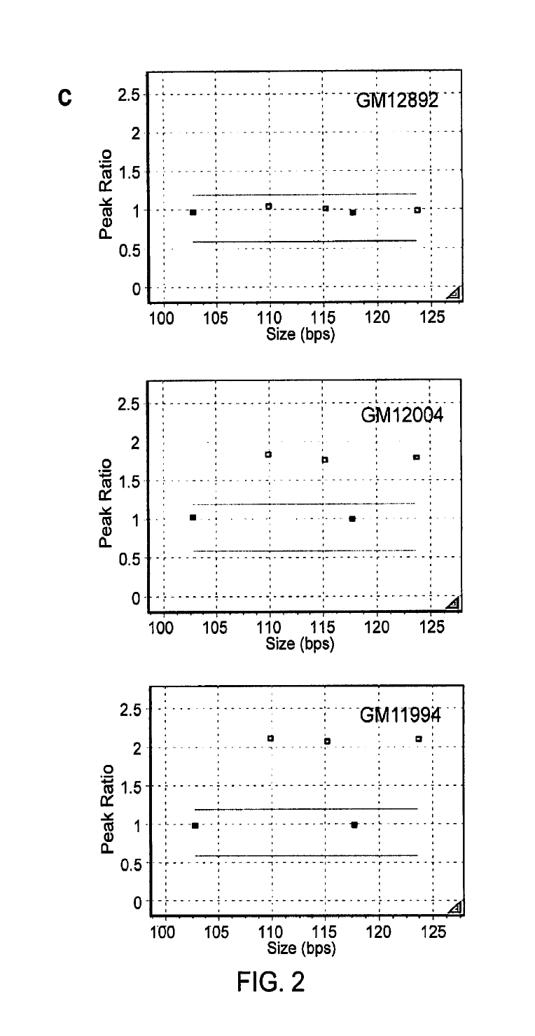

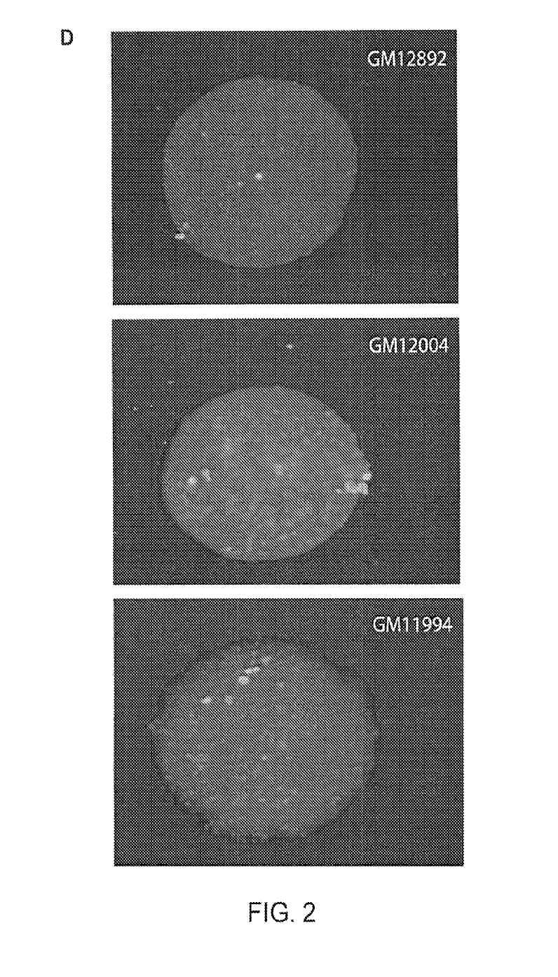

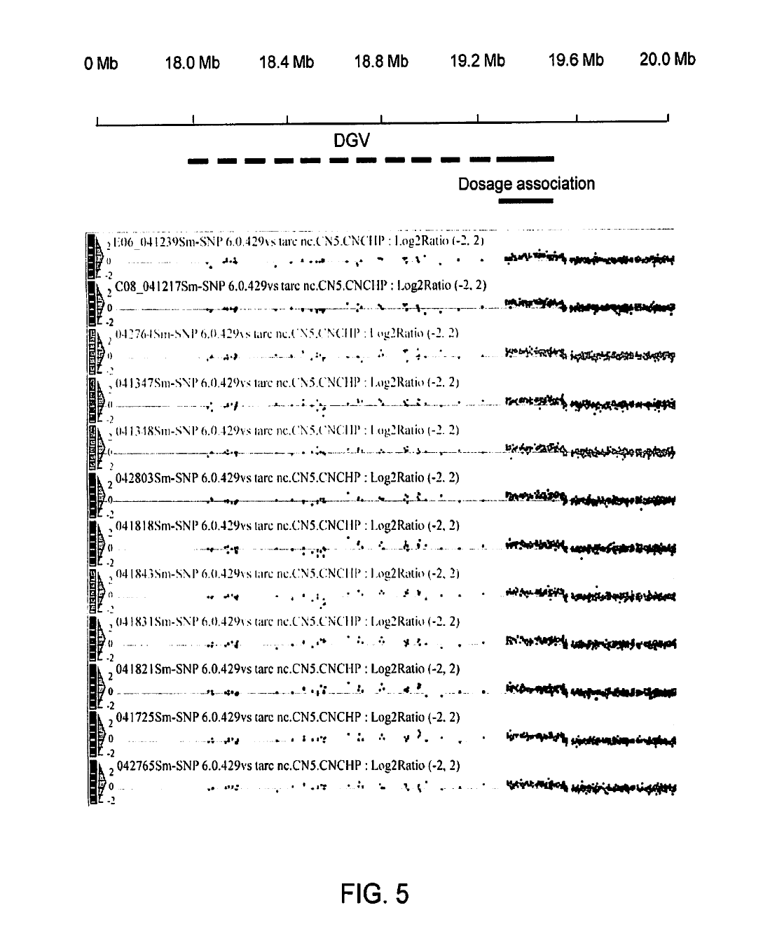

A cluster of hazard regression results was identified displaying significant association with AAO on chromosome 14 in the pericentromeric region of the q arm. In this region there were 22 results with a −log_e p value greater than 7 and with the same direction of effect between log_{—}2-ratio and AAO (FIG. 1A). The region on chromosome 14 also appears to have high variability in CN state in our patient cohort so that the association is not driven by a single outlying individual or few individuals. The significant results identified by regression are among the highest 1% of allelic variability across all genomic regions (FIG. 1B). The array data heatmap (FIG. 1C) visually demonstrates the copy number state in conjunction with the AAO data (FIG. 1C). The high copy number state (defined by a mean log_{—}2-ratio for this region greater than zero) is correlated with younger AAO with a median of 67 years while the low copy number state (mean log_{—}2-ratio less than zero) with later AAO and a median of approximately 77 years; together these values suggest a difference in median AAO of approximately 10 years for these two classes. The region detected corresponds to a multiallelic cluster of overlapping CNVs at the 14q11.2 locus with various breakpoints (FIG. 2A and FIG. 5). The association signal comes from the region harboring OR4M1, OR4N2, OR4K2, OR4K5 and OR4K1 (respective but exemplary GenBank® Accession numbers are NM_{—}001005500; NM_{—}001004723; NM_{—}001005501; NM_{—}001005483; NM_{—}001004063, all of which are incorporated by reference herein in their entirety). The summary test statistics for the region are summarized in Table 3.

TABLE 3
Cox proportional hazard outcomes in
the discovery and replication cohorts

| Cohort | Model |
|---|---|
| Discovery | Dosage |
| Replication | Dosage |
| Replication | Dosage |
| Replication | Categorical |
| Meta Analysis | Dosage |
| Meta Analysis | Dosage |
| Meta Analysis | Categorical |

Categorical model: The data suggests that the majority of the signal originates from the 5+ state. A categorical model was applied (category 1 includes 2, 3 and 4 CN states, category 2 harbors the 5+ CN state) to estimate the attributable risk for the 5+ state. The genetic models consistent with this analysis include risk conferred by lack of normal allele, the triplication allele being dominant risk allele or a dosage threshold effect.

The complete set of hazard function outcomes with −log_e p>7 depicted in the Manhattan plot are summarized in Table 4.

TABLE 4
Demographic data for the discovery and replication cohorts and the control subjects

| | | Sex | | | | | APOE | | |
|---|---|---|---|---|---|---|---|---|---|
| | No. of | ratio | AAO (yr) | | Age (yr) | | N/N | N/4 | 4/4 |
| | subjects | F:M | mean ± SD | range | mean ± SD | range | n(%) | n(%) | n(%) |
| Discovery | 40 | 28:12 | 70.5 ± 10.2 | (50-84) | 77 ± 9.7 | (58-92) | 35 (88) | 5 (12) | 0 (0) |
| Replication | 507 | 308:199 | 71.3 ± 9 | (47-98) | 77.5 ± 8.9 | (52-103) | 189 (37) | 249 (49) | 69 (14) |
| Control | 78 | 50:28 | NA | NA | 72.5 ± 8.8 | (57-94) | 49 (63) | 27 (35) | 2 (2) |
| Metaanalysis | 547 | 336:211 | 71.3 ± 9.1 | (47-98) | 77.5 ± 9 | (52-103) | 234 (43) | 254 (46) | 69 (13) |

### Example 4

### QC and Genomotyping Consistency Between Platforms and Fish Confirmation

QC measures are detailed below. The CNVs inferred in the replication cohort are depicted in FIG. 2A in the context of previously observed variants in the Database of Genomic Variants. The sizes and location of the calls inferred in the replication study are in agreement with the reported CNVs. The differences could be related to the various platforms applied or the disease specific cohort. Gene dosage inference was validated and was congruent between the Agilent 244k, the Affymetrix arrays and the MLPA assay by performing all pairwise combinations of the genotyping assays on a subset of samples. CN state was concordant between Affymetrix-Agilent, Affymetrix-MLPA and Agilent-MLPA in 97, 89 and 92% of samples, respectively. FIG. 2B depicts the correlation between the CN call on the Affymetrix array and the log_{—}2-ratio on the aCGH. MLPA confirmed the absolute CN states.

FISH was utilized to estimate absolute dosage and to localize the additional copies of the genetic regions of interest. Because cell lines are not available on the subjects studied here, the HapMap CEU samples (GM12892, GM11994 and GM12004) were utilized for which Affymetrix data (see the Affymetrix website) and cell lines (see the HapMap website) are publicly available. Samples with 2, 3 or 4 copies of the most common allele were selected for FISH, which confirmed the presence of 2, 3 and 4 copies in the corresponding samples, respectively (FIG. 2D). The FISH indicates that up to 3 copies are located on the same chromosome in close proximity (GM11994).

### Example 5

### Olfactory Receptor Cluster CN Association with AAO of AD in the Replication Cohort

The Cox proportional hazard analysis confirmed the association of CNV Variation_{—}0316 (see Database of Genomic Variants on world wide web) dosage with AAO of AD (p=0.03) in the replication cohort (Tables 3 and 5).

TABLE 5
Summary of AAO distribution in genoptype groups

| Genotype | Patients | | | AAO | | |
|---|---|---|---|---|---|---|
| CN | APOE4 | (N = 507) | Median | Mean | SE | range |
| 2 | N/N | 80 | 73.5 | 73.19 | 1.17 | 48-98 |
| | N/4 | 110 | 73 | 71.95 | 0.74 | 52-89 |
| | 4/4 | 29 | 65 | 66.55 | 1.4 | 48-80 |
| 3 | N/N | 78 | 76 | 73.04 | 1.07 | 47-86 |
| | N/4 | 80 | 74 | 72.11 | 0.95 | 51-89 |
| | 4/4 | 26 | 69 | 69.21 | 1.4 | 50-86 |
| 4 | N/N | 28 | 69 | 69.33 | 2.06 | 52-90 |
| | N/4 | 47 | 72 | 71.33 | 1.22 | 49-90 |
| | 4/4 | 9 | 66 | 65.73 | 1.53 | 58-73 |
| 5+ | N/N | 2 | 65.5 | 65.5 | 0.5 | 65-66 |
| | N/4 | 13 | 67 | 66.35 | 2.43 | 53-83 |
| | 4/4 | 5 | 58 | 62 | 3.91 | 55-76 |

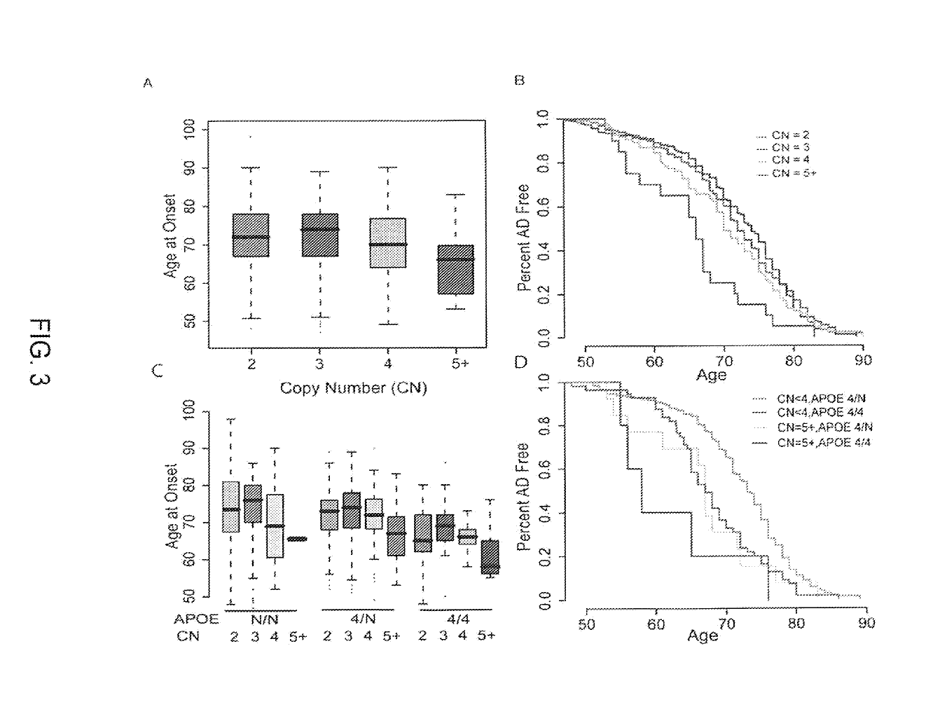

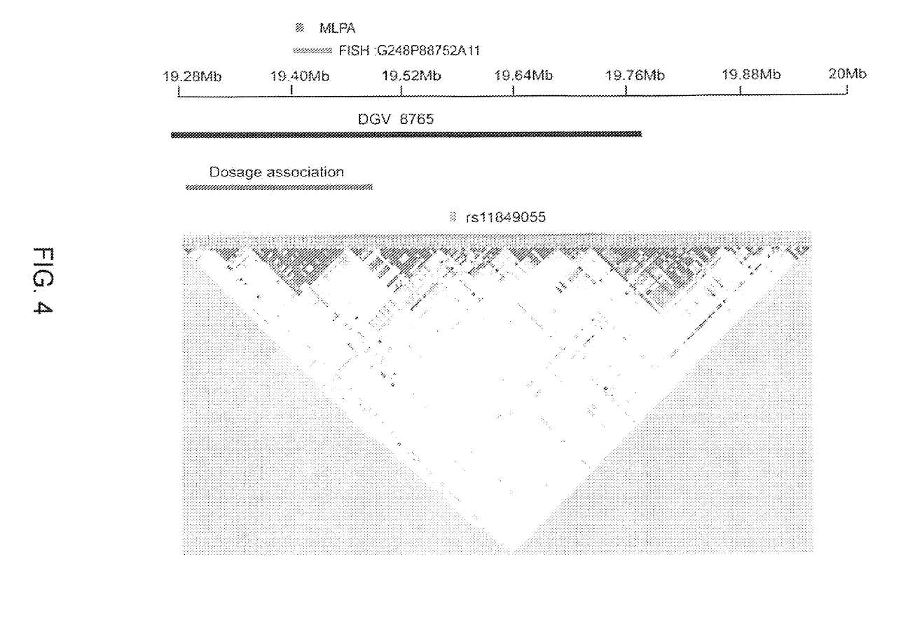

The analysis also replicated significant AAO association of APOE 4/4. No interaction between APOE and the CN locus was identified by the regression; no gender effects were detected. The dosage association of the olfactory receptor gene cluster on AAO of disease is presented in the boxplot (FIG. 3A). The time to event survivorship curves for the various copy number states depicts the percentage of subjects diagnosed by age for each copy number state (FIG. 3B). The association of the 14q locus appears to be independent of and in addition to the effect of APOE. In addition, the inventors calculated OR for developing AD by 72 years of age (median AAO of the complete dataset) for the CN5+ state and the APOE4/4 genotype. The OR for the CN5+ and the APOE4/4 genotype was 5.8 (CI 1.7-20) p=0.001 and 4.1 (CI 2.3-7.6) p<0.0001, respectively. The most striking association signal appears to originate from the multi-copy gain state on all APOE backgrounds; the 2 copy and 3 copy states have similar AAO and the 4 copy state has a trend toward earlier AAO on all APOE4 backgrounds. The inventors built an additional Cox proportional hazard model treating the 2, 3 and 4 copy states as a single class and the 5+ state as the other CN class based on this observation (RR=2.05, CI(1.33-3.18), P=0.001) (Table 3). In this model, either the lack of a normal allele or the triplication allele confers the risk, in certain embodiments. Subjects with four copies are a mixed population harboring no normal allele (2+2) or are heterozygous for the triplication (1+3).

### Example 6

### Exemplary Supplementary Materials and Results

Exemplary methods and results are described in this example.

### Methods

### Subject Cohorts

Over 90% of subjects in the discovery cohort were Caucasians and APOE4 non-carriers. The replication cohort included 507 Caucasian subjects. A control population consisting of 78 Caucasian normal subjects over age 55 were also assayed for generating a reference file for the CN analysis. Controls were recruited at each participating site by the same inclusion criteria, including age over 55 years, male and female, unrelated to AD subjects, CDR global score 0, normal performance on activities of daily living, and all information was obtained from surrogate historian. After enrollment all control subjects underwent neuropsychological testing including assessment of Global cognitive functioning/status (MMSE and CDR), Attention (Digit Span and Trails A), Executive function (Trails B and Clock Drawing; Texas Card Sorting is optional), Memory (WMS Logical Memory I and WMS Logical Memory II), Language (Boston Naming and FAS Verbal Fluency), Premorbid IQ (AMNART), Visuospatial Memory (WMS-Visual Reproduction I and II), Psychiatric (Geriatric Depression Scale; Neuropsychiatric Inventory-Questionnaire) and Functional (Lawton-Brody ADL: PSMS, IADL). Control subjects showing impairment were excluded from the control cohort after consensus review.

Informed consent was obtained from all subjects prior to inclusion. Genomic DNA was isolated from whole blood by the Puregene DNA isolation kit (Qiagen) according to the manufacturer's instructions.

### APOE Genotyping

Genotyping was performed according to manufacturer's instruction with real-time PCR using custom TaqMan probes (Applied Biosystems, Inc) unique for SNPs of rs7412 and rs429358 at nucleotides 112 and 158 of the APOE gene, respectively. All amplifications were carried out in an ABI 7900HT thermal cycler (Applied Biosystems, Inc; Foster City, Calif.). APOE genotype was determined from the combination of alleles present at the 112 and 158 polymorphisms.

### Detection of Copy Number Variation and Test of Association in the Discovery Cohort

The experimental procedures on the Agilent 244 k array were performed according to the manufacturers' instructions. The QC metrics for the aCGH experiment required the Agilent QC metric derivative log_{—}2-ratio spread (dlrs) to be less than 0.4. Normalized log_{—}2-ratio data were generated by the manufacturer's microarray scanner and quantification software (CGH analytics, Agilent) and were sorted by genomic location using the hg18 build of the human genome (build 36.1). The position ordered data were grouped into 5-probe sliding window groupings (bins) of adjacent oligonucleotides; the mean log_{—}2-ratio for each bin was determined for use in the subsequent AAO analysis. The 5-probe sliding window size was selected empirically based on extensive clinical genotyping experience, which utilizes confirmation by fluorescent in situ hybridization (FISH), suggesting 5 consecutive probes as stable detection threshold for CNV events as opposed to individual oligonucleotides which can give more variable signal.

Hazard function regression was performed using the survival package in R (see The Comprehensive R Archive Network on the world wide web) with AAO as the outcome variable and the 5 oligonucleotide bin mean log_{—}2-ratios as the explanatory variable under a parametric Weibull model for the AAO times. This model treats the log hazard as a linear function of the bin mean log_{—}2-ratios. In addition, the inter-subject allelic variation of each 5 oligonucleotide window was calculated across the cohort by computing the variance of the mean log_{—}2-ratio across subjects to provide an additional filter of allelic heterogeneity of each bin.

A preliminary AAO association filter for the discovery cohort was defined by using allele frequency of variants in all CNV regions. With a sample size of 40, all allele frequency >0.05 (at least 2 events per cohort) were required to be considered as an association signal. The inventors empirically determined the number of common CNV regions in the cohort by segmenting the data using the ADM2 algorithm implemented in the DNAanalytics software (Agilent). There were 107 and 58 CNV regions with allele frequency >0.05 and >0.075 in the discovery cohort, respectively. Using these values as an estimate for number of comparisons, a heuristic Bonferroni correction was calculated to achieve an alpha of 0.05 and 0.1 (−log_e (0.05/58)=7.05 and (−log_e (0.1/107)=6.975). Thus the preliminary AAO association filter was defined by using −log_e p-value threshold of 7 for the hazard regression coefficient for the CN value.

### CNV Genotyping by Genome-Wide Human SNP Array 6.0

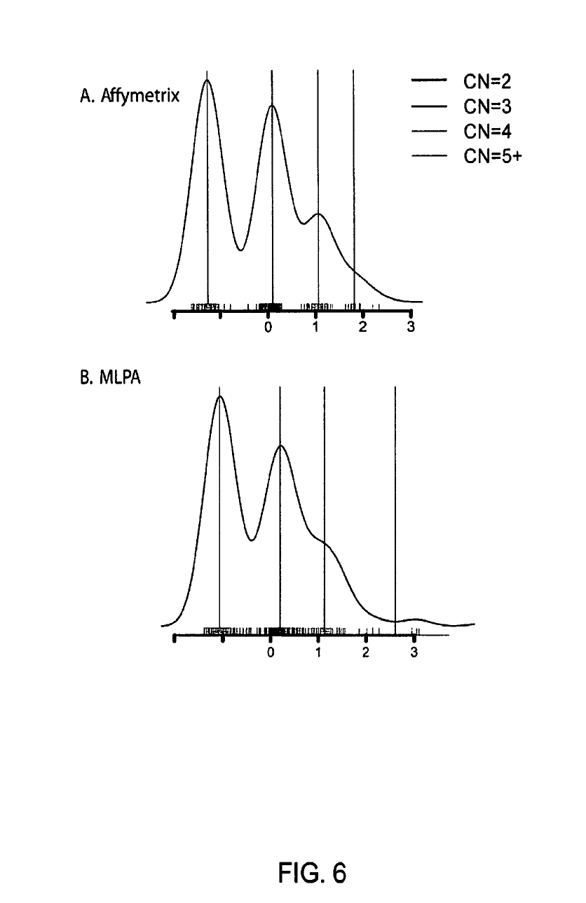

Array based genotyping for the replication cohort was performed on the Genome-Wide Human SNP Array 6.0 (Affymetrix) according to the manufacturer's instructions. CNV analysis was performed in the Genotyping Console™ Software (see the Affymetrix website). QC measures for the Genome-Wide Human SNP Array 6.0 (Affymetrix) array included contrast QC (>0.4) and Median of the Absolute values of all Pairwise Differences (MAPD)<0.4. As this software is unable to distinguish CN states over 4, a Gaussian mixture model applied to the normalized mean intensities was used to assign CN states. To make these mixture model based inferences, a mean value was computed for each individual averaging across the probe values. These data were normalized by subtracting the cohort mean and dividing the mean centered values by the standard deviation across the cohort. The resulting normalized data have a single value representing the genotype data for each person and these normalized values collectively have mean 0 and variance 1. The R package cluster and the method PAM (Pardoning About Medoids) were used to robustly partition the data into 4 allelic classes. The mean and variance of each allelic class was estimated based on the PAM classification. The normalized data was processed through a univariate Gaussian mixture classification procedure using the PAM clustering result to determine an initial estimate of the means, variances and genotype frequencies for each CN state. The prior means were −1.195, 0.1, 1.04, and 1.84 for CN states 2, 3, 4 and 5 respectively. The standard deviations were 0.18, 0.20, 0.20 and 0.30, respectively; the estimated genotype frequencies were 0.42, 0.34, 0.186 and 0.054 for CN states 2, 3, 4 and 5, respectively. This empirical mixture model classification procedure served to confirm the PAM clustering calls and to estimate posterior probability for each individual call. See FIG. 6 for a visualization of this data and the genotype assignments together with the mean assay values for each genotype class. These calls were cross-validated with direct information from MLPA results on a subset of cases and all of the 5+ calls (FIG. 5).

### Multiplex Ligation-Dependent Probe Amplification (MLPA) Assay

The MLPA¹⁶ assay for OR4K2 region was designed to verify the CNV that was detected from aCGH and Genome-Wide Human SNP Array 6.0 (Affymetrix). The assay was performed with SALSA EK kit (MRC Holland, Amsterdam, The Netherlands) and a custom designed probe set according to the manufacturer's “DNA Detection/Quantification” protocol. Three probes were designed within the OR4K2 gene and 2 reference probes (LAT and MAZ) for other genes located on a different chromosome. Exemplary probe sequences are depicted in Table 6.

TABLE 6
Exemplary probe sets of the MLPA assay

| Primer name | Sequence |
|---|---|
| OR4K2-1 - LPO | GGGTTCCCTAAGGGTTGGAGTGGGTAACAGCCTCATAGTCATCACAGTTATAGTGGAC |
| | (SEQ ID NO: 1) |
| OR4K2-1 - RPO | CCTCACCTACACTCTCCTATGTATTTCCTGCTTACCTCTAGATTGGATCTTGCTGGCAC |
| | (SEQ ID NO: 2) |
| OR4K2-2 - LPO | GGGTTCCCTAAGGGTTGGACTTCCTGGATTATGGGAGTTATGCATTCAATGAGTCAG |
| | (SEQ ID NO: 3) |
| OR4K2-2 - RPO | GTCATATTTGCCCTCACGTTACCATTCTGTGTCTAGATTGGATCTTGCTGGCAC |
| | (SEQ ID NO: 4) |
| OR4K2-3 - LPO | GGGTTCCCTAAGGGTTGGACAGCTCATTTCATTGTTGTCTTCTTGTTCTTTGGGCCATG |
| | (SEQ ID NO: 5) |
| OR4K2-3 - RPO | CATCTTCATCTACATGTGGCCACTAAGCAGCTTTCTCACAGACTCTAGATTGGATCTT |
| | GCTGGCAC (SEQ ID NO: 6) |
| LAT - LPO | GGGTTCCCTAAGGGTTGGACCTGCTGCTGCCCATCCTGGCCATGTTGATG |
| | (SEQ ID NO: 7) |
| LAT - RPO | GCACTGTGTGTGCACTGCCACAGACTGCCAGTCTAGATTGGATCTTGCTGGCAC |
| | (SEQ ID NO: 8) |
| MAZ - LPO | GGGTTCCCTAAGGGTTGGACTCGGCTTATATTTCGGACCACATGAAGGTGCACAG |
| | (SEQ ID NO: 9) |
| MAZ - RPO | CCAGGGTCCTCACCATGTCTGTGAGCTCTGCAACAAAGGTACTCTAGATTGGATCTT |
| | GCTGGCAC (SEQ ID NO: 10) |

The SALSA Q+D control fragments (MRC Holland) were used for quality control purpose to assess if input DNA quantity and ligation reaction were adequate. The probe mix consisted of 0.8 pmol of each custom probe and 24 μl of SALSA Q+D control mix diluted to a total volume of 600 μl with TE. Completed MLPA reaction was diluted 1:20 in water, and 1 μl of each diluted product was combined with 9 μl of GeneScan 500 LIZ Size Standard (Applied Biosystems, Foster City, Calif.) and Hi-Di formamide mix. The MLPA products were run on a 3730×1 DNA Analyzer (Applied Biosystems) using ABI Foundation Data Collection software V3.0, and data were analyzed using GeneMarker software V1.51 (SoftGenetics, LLC, State College, Pa.). The custom assay was validated using Hapmap samples (GM12892, GM12004 and GM 11994). To make the MLPA procedure more quantitative and robust, the same PAM clustering was applied followed by empirical Gaussian mixture classification used for the Affymetrix data to the MLPA mean probe intensities. In this case the prior means for the genotype classes for the normalized (mean 0, variance 1) MLPA values were −0.99, 0.19, 1.20, 2.48 for CN states 2, 3, 4 and 5 respectively. The standard deviations were 0.19, 0.21, 0.23, and 0.65. The prior genotype frequencies were 0.43, 0.35, 0.17 and 0.04. A visualization of this data and the genotype assignments for both cases is provided in FIG. 6.

### QC Measures

In the discovery cohort, all Agilent array experiments passed the Agilent dlrs threshold. In the replication cohort, 14 samples failed Affymetrix contrast QC, 2 samples failed MAPD and 42 samples failed because of intensity data distribution resulting in number of CNV calls more then 2SD of the mean. 243 Affymetrix arrays passed QC. In the replication cohort all samples passed the MLPA QC.

### Genomotyping Consistency Between Platforms

Crossvalidation of the three genotyping methods was performed by genotyping a large number of samples with at least two methods. Genotyping was performed by Affymetrix and Agilent in 35 samples (concordance 97%), Affymetrix and MLPA in 18 samples (concordance 89%) and Agilent MLPA in 25 samples (concordance 92%). Breakpoint correlation was confounded by the differential coverage of the genomic region between the two platforms.

### Fluorescence In Situ Hybridization (Fish)

Metaphase spreads were prepared from colcemid (10 ug/ml)-stimulated human lymphoblast cell cultures GM12892, GM12004 and GM11994 (Coriell). Fosmid (G248P88752A11) and BAC (RP11-52401) clones were chosen from the physical maps of the regions of interest using the UCSC Browser (see world wide web) and obtained from the Human Genome Sequencing Center of Baylor College of Medicine. FISH was performed according to a modified procedure of Stankiewicz et al. (2001). Briefly, Fosmid and BAC cloned DNA was isolated using the Plasmid DNA Purification kit (Qiagen), and 200 ng probes were labeled with biotin or digoxigenin using nick-translation reaction (BioNick Labeling System, Invitrogen; DIG-Nick Translation Mix, Roche) and visualized with FITC avidin (Vector) or rhodamine-labeled antibodies (Sigma). The same stringency conditions were used for all experiments, i.e. hybridization with 3.5 μg Cot-1 DNA (Gibco BRL) and 25 μg salmon sperm DNA (Sigma) at 37° C. in 50% formamide, 2×SSC, 10% dextran sulfate, pH 7.0; washing for 15 min at 42° C. in 3 changes of 50% formamide/2×SSC followed by 15 min at 42° C. in 2×SSC. Chromosomes were counterstained with DAPI (Sigma). A Zeiss Axioplan2 epifluorescence microscope with suitable filter set and high-resolution CCD camera (KAF 1400, Photometrices) was used for capturing images.

### Population Substructure

The complete SNP and CNV dataset ascertained from the Genome-Wide Human SNP Array 6.0 (Affymetrix) was used to assess the possibility of population substructure confounding CN state (N=243). The population substructure analysis was performed by principal component analysis with the Eigenstrat package for the first 10 principal components (Price et al., 2006). The projected values for the samples were then plotted against each other for the first 10 components.

### Results

### Sample Characteristics

Cohort characteristics are summarized in Table 5. Mean AAO in the discovery and replication cohorts were 70.5 (range 50-84) and 71.3 (range 47-98), respectively. The Spearman correlation between the two methods of AAO phenotyping (caregiver estimate by prompted standard questions regarding onset of symptoms compared with physician estimate of duration of illness using structured interview with landmark event to facilitate recall (Doody et al., 2004) was well correlated (Spearman correlation rho=0.9409, p=2.2×10⁻¹⁶; CI 0.90-0.97). The study design is cases-only; a set of control samples was used to compute the reference genome for the CN analysis (Methods). The mean age of individuals used as controls was 72.5 (range 57-94).

### Population Substructure

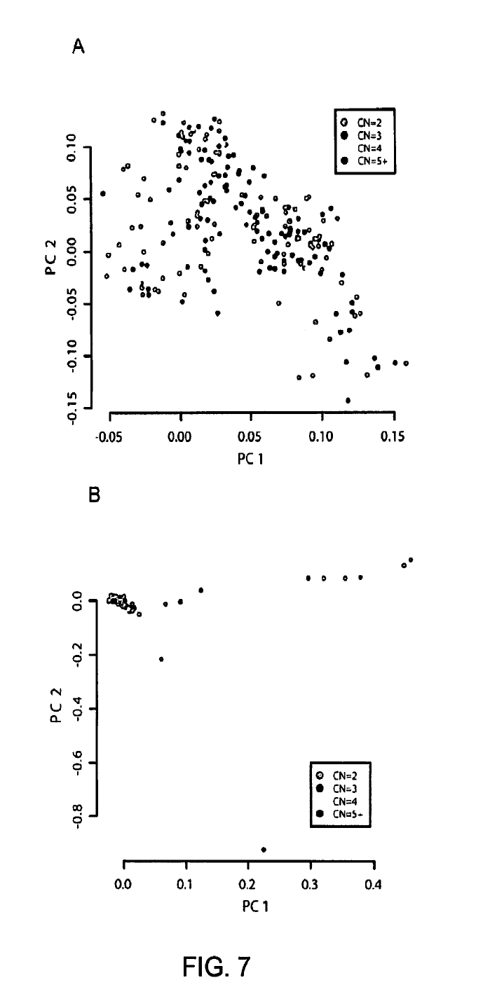

Analysis to consider population substructure was performed using principal component analysis (PCA) for both the SNPs and CNVs concomitantly ascertained on the Genome-Wide Human SNP Array 6.0 (Affymetrix) for the AD subjects (cases-only association analysis). The principal components for the top ten eigen values were plotted pairwise for the SNP and CNV dataset. FIGS. 7A and 7B show an absence of pattern between the various copy CN states and the first two principal components for both the CN and the SNP datasets, respectively. Thus, the CNV PCA excluded the possibility of spurious association caused by systemic effect on the individual CN states, and the SNP PCA confirmed that the CN states are not a result of population substructure and admixture.

### Example 7

### Significance of Certain Embodiments of the Invention

AAO was estimated by two methods, the single question inquiry and a validated structured interview with landmark event to facilitate recall of the surrogate historian (Doody et al., 2004). The Spearman correlation of the two methodologies was significant (Spearman correlation rho=0.9409, p=2.2×10⁻¹⁶; CI 0.90-0.97) and outlier and residual analysis showed no systematic deviation between the two methods. The inventors have undertaken a cases-only copy number variation genome wide association study with AAO of AD and have detected association of gene dosage of the olfactory receptor region on chromosome 14q11.2 with a substantial association (9.75 years difference in median AAO of AD). The replication cohort confirmed the association of the inferred CNV locus with AAO of AD. Moreover, this analysis showed the CN association to be independent of and in addition to the association observed for APOE4. The CNV contributing to the association signal harbors an olfactory receptor gene cluster, which contains plausible candidate genes in disease pathomechanism in agreement with clinical and neuropathological observations. The allele frequencies of the CNV region identified are comparable to prior reports (Young et al., 2008), and the allele frequencies in the replication study are consistent with detection of the association between CN and AAO in the discovery cohort. Applying a categorical model to the replication dataset yielded a more significant association, suggesting that lack of normal allele or the triplication allele confers the highest risk.

Previous studies (Li and Fan, 2000; Scott et al., 2003; Bertram et al., 2003) investigated the genetics of AAO of AD used SNP datasets, and thus the CNV approach is complementary to these studies. The prior SNP association studies are likely to have lacked sensitivity to detect this locus because of its complex allelic structure. While the ability of SNPs to tag CNVs is debated (Conrad et al., 2007), multiallelic loci with rare individual allele frequencies are likely less amenable to SNP tagging. The Cox proportional hazard regression was performed on 148 SNPs flanking the CNV locus using additive, recessive and dominant models with the assumption that if SNPs are in LD with the CNV one would detect the association (data not shown and FIG. 5). Only one SNP (rs11849055) reached Bonferroni corrected significance in the recessive model, and this SNP is not in LD with the CNV. The results indicate that in certain embodiments of the invention the observed CNV events likely occurred on various alleles as independent events. While the aCGH, the SNP arrays and MLPA assay all measure gene dosage, they do not provide positional or orientation information. The absolute dosage and the location of the gain by FISH was confirmed for a subset of alleles using HapMap cell lines with corresponding dosages (FIG. 2D).

In some embodiments of the invention, the invention shows an association of CN and AAO, and does not lead one to causal genes per se. However, the association signal arising from a multiallelic CNV locus and the gene dosage association indicate that the gene content of the CNV including the olfactory receptor cluster is a modifier of AAO in AD, in certain aspects of the invention. Prospective cohort studies established the risk olfactory deficit confers for the development of cognitive decline. A prospective study of 1920 cognitively normal subjects found a significant association between olfactory impairment at baseline and 5-year incidence of cognitive impairment (OR=6.62, CI=4.36-10.05) (Schubert et al., 2008). Another study of 1,604 non-demented older adults found that women with anosmia who possessed at least 1 APOE4 allele had an odds ratio of 9.71 for development of cognitive decline over the ensuing 2 years, compared with an odds ratio of 1.90 for women with no olfactory dysfunction and at least 1 such allele (Graves et al., 1999). Neuropathological series have found marked cell loss in the olfactory bulb and anterior olfactory nucleus and the presence of disease-related pathology: neuritic plaques and neurofibrillary tangles in these structures (Ohm and Braak, 2007). Whether loss of olfaction is a primary or secondary phenomenon in the pathomechanism of AD is unclear, however this genetic observation raises the possibility for its role in modifying the AAO.

Although the allele frequency is low, in a disease with a prevalence of 5.3 million a frequency of 4% translates to over 200,000 subjects with the high copy number state affected by the disease. AD patients with the earliest AAO represent the most desired group for intervention with disease modifying therapy. Pathogenic CNVs are more amenable to therapy than other types of genetic variation, in specific embodiments of the invention. In contrast to mutation events resulting in loss of function or toxic gain of function, CNVs alter gene dosage, allowing modification by small molecules which may offset the dosage effects (Lupski, 2007). For example, the CMT1A duplication rat model treated with a progesterone antagonist achieved clinical and pathological improvement even by partial correction of gene expression by epigenetic modification (Sereda et al., 2003). In certain embodiments, evaluation of olfactory receptor copy number leads to both earlier detection and disease modifying therapy, in specific embodiments of the invention, both of which have a major impact on the public health burden of AD.

Here, it is recognized that copy number variants CNVs are important mechanisms of genetic variation contributing to disease phenotypes. In this cases-only genome-wide CNV association study looking for loci affecting AAO of AD a chromosomal segment on 14q11.2 (reference sequence position 19.3-19.5 Mb) was identified where gene dosage is associated with AAO of AD (genome-wide adjusted p<0.032). The association was replicated in an independent cohort of 214 subjects using independent experimental and statistical methods (see above). Interestingly, this region encompasses a cluster of olfactory receptors, shedding light onto the role of olfactory dysfunction in AD. This results indicate that Olfactory Receptor-CNVs or patterns modify AAO of AD. This is based on the following observations. First, there is clinical evidence that there is olfactory dysfunction in patients with AD and that impaired olfaction at baseline in cognitively normal elderly subjects correlates with cognitive impairment at 5 year follow-up. (Devanand et al. 2000; Djordjevic et al. 2008) (Mesholam et al. 1998) Second, the neuropathological involvement of the olfactory pathway in AD and its neuroanatomical connections to the structures involved in AD and memory raises the possibility of a role of this gene family in disease mechanism. (Eibenstein et al. 2005) Third, olfactory receptors (ORs) are highly variable in copy number and olfaction has been implicated with aging with multiple lines of evidence in several model organisms. (Young et al. 2008) Fourth, in addition to the direct involvement of the olfactory pathway in AD, expression of olfactory receptors is not limited to the olfactory neurons; rather, various patterns of expression are present in the entorhinal and temporal cortices, raising the possibility of a functional role for the ORs outside the olfactory neurons (Feldmesser et al. 2006). In an embodiment of the invention an olfactory gene is assayed to determine the CNV. In a specific embodiment of the invention a higher CNV indicates a lower AAO of AD.

### Example 7

A custom array for comparative genome hybridization (aCGH) is made covering all OR genes to comprehensively study the ORs. The array approach allows complete coverage of all of the OR regions in order to establish their modifier effect on AAO. This Example provides data for addressing the predictive value of the modifiers in prospective cohorts of patients with MCI. 600 subjects (500 AD and 100 normal controls) have been enrolled. Baseline neuropsychological measures (detailed in preliminary data) were performed and entered into the TARC database. Subjects consented to genetic testing and DNA was extracted and banked. The protocol will be amended with the olfactory phenotyping. The olfactory genotype and phenotype are correlated using the aCGH and the University of Pennsylvania Smell Identification Test (UPSIT) measure. Cognitive profiles are compared at presentation between the various OR copy number states to assess whether there is a cognitive endophenotype associated with the OR genotypes.

Potential genetic pathomechanisms of the modifier effect are elucidated. Olfactory receptor expression is regulated by allelic exclusion: only a single allele of a single receptor is expressed in an olfactory neuron. The proper connections of the olfactory receptors to the olfactory glomeruli depend upon the allelic exclusion. The copy number variation may mimic biallelic expression perturbing the connections in the olfactory pathway. OR expression patterns in the temporal lobe of AD brains were studied in the examples above. The expression experiment is expanded to human olfactory bulb; RT-PCR is developed for transcripts of subthreshold abundance for array-level detection. If CN variant OR expression is detected, laser-captured single-cell expression analysis is performed and the SNPs within the sequence are used to distinguish whether one or two copies of the OR on the duplicated allele is expressed to address whether the duplication may mimic biallelic expression.

Coverage of the OR Regions on Affymetrix and Agilent Platforms and Array Design.

The coverage of the OR regions on the 244k Agilent aCGH and the Genome-Wide Human SNP Array 6.0 (Affymetrix) platform was assessed. The OR gene genomic coordinates from the manuscript Nozawa et al were collated (Nozawa et al. 2007). These genomic coordinates were mapped to the build 36 assembly. Subsequently, the Agilent 244 k and Genome-Wide Human SNP Array 6.0 (Affymetrix) array libraries were screened for probes that are located in these regions. The Agilent and Affymetrix array cover 14% and 49% of the OR genes with at least 1 probe, respectively. As the dynamic range of these assays do not allow single probe resolution the true detection coverage is lower.

Expression of OR Genes in Temporal Lobe of AD Patients and Controls.

Illumina array (human WG-6 expression Beadchip) were performed on 27 AD and 15 normal control post mortem frozen temporal lobe tissue. The samples were obtained from the BCM/Methodist Brain Bank and the New York Brain Bank. All samples were deidentified. The AD samples were diagnosed with definite AD by a neuropathologist. The control specimens were reviewed by a neuropathologist and the diagnosis of no pathological change was assigned. For RNA preparation, brain tissue was homogenized with a tissue homogenizer in Trizol (Invitrogen) following standard procedures and further purified with Rneasy mini kit (Qiagen). Illumina array (human WG-6 expression Beadchip) experiments were performed by the manufacturer's instructions. Array quality control parameters included 50% of transcripts detected, expression over background p<0.01 and pairwise concordance r²>0.8. Five arrays (3 AD and 2 controls) were excluded. 15 various ORs were detected above background with p>0.01 in the 37 arrays with RNA Integrity number (RIN) 5.2-9.0 (7.13±1.19). Considering the cellular heterogeneity and the likely subthreshold abundance of transcript for the OR genes, the detection by array suggests that other ORs may also be expressed in the temporal lobe and other brain areas.

Study Cohort (TARC Cohort)

The TARC cohort are used (Table 3) as i) subjects have been recruited and are being longitudinally followed, ii) clinical and neuropsychological evaluations have been performed and databased, iii) AAO is ascertained by standardized methods across sites, iv) DNA was extracted from blood to avoid tissue culture artifacts (de novo CNVs due to culturing) and is available, v) genotype data on the same subjects is available (for population substructure and admixture analysis) and vi) existing TARC infrastructure can be built on.

The TARC cohort is comparable to other ongoing research cohorts (ADNI, NIA LOAD, Alzheimer's Disease Centers Consortium and Framingham) with its inclusion and exclusion criteria are more in depth regarding AAO phenotyping by using two methodologies. Another advantage is the blood derived DNA which overcomes the concerns relating to de novo CNV events caused by tissue culturing. The NIA LOAD study ascertains AAO by the single question method thus may serve as a replication cohort in the future. The Framingham has prospective AAO information, but the number of subjects converting to AD provides only a small sample size.

The TARC has so far recruited 521 individuals with a diagnosis of AD and onset of symptoms at age 55 years or above and 228 normal controls and is in the process of adding 100 AD, 200 controls, 250 MCI and 500 Hispanic AD subjects, MCI and controls over the next two years. Genotyping is ongoing for the first 600 subjects using the Affymetrix 6.0 platform.

Cohort Characteristics

AD Patients

Inclusion Criteria
1) Men and women 55 years of age or older with a diagnoses of Probable AD (NINCDS-ADRDA criteria)
2) Age at onset of dementia by both single question and structured interview is recorded
3) Subjects must have MMSE score ≧11

Exclusion Criteria:

Patients with a Hachinski Ischemic Score >4 or history of major cortical infarction, diagnosed either by neuroimaging, or clinical stroke with persistent focal neurologic deficit were excluded.

Control Participants

Inclusion Criteria
1) Men and women 55 years of age or older
2) CDR=0 (Global Score)
3) Normal cognition based on reliable informant report
4) Normal cognitive capacity to perform independent activities of daily living based on reliable informant report
5) No active or uncontrolled CNS, systemic, or psychiatric condition that would affect cognition based on referring physician's report (preferred) or reliable informant report
6) No use of psychoactive medications in amounts that would be expected to compromise cognition or for reasons indicating a primary neurologic disease or psychiatric illness based on referring physician's report (preferred) or reliable informant report
7) All information must be Informant-based (provided by someone other than the person being recruited) with reliability of informant based on judgment of examiner

Exclusion Criteria
1) Known relation to a patient in the TARC study
2) History of dementia, definite stroke (clinical or imaging), movement disorder, MS, brain tumor, seizures, severe head trauma, schizophrenia, bipolar disorder, major depression

Procedures

Informed Consent

All subjects with Alzheimer's disease were recruited with consent of a legal representative. Once contact with the legal representative or non-AD controls had been made, the consent process began. No cognitively impaired subjects were included without the proper informed consent of a legal representative who maintains that subject's best interest.

Recruitment of Subjects

All subjects have been recruited for the genotyping and cognitive testing. Recruitment for the olfactory phenotyping will be accomplished at the time of the annual follow-up visit by reconsenting the subjects. The new consent form will contain specific questions whether the subject consents to sharing samples, coded clinical and genotype information with other investigators through NCRAD and NIAGADS (or another NIA approved repository). IRB protocols at each site will be amended for the olfactory phenotyping and data sharing. Coordinators at each site will administer the UPSIT measure. The UPSIT measure will be ascertained at the time of the annual follow-up visit.

Sample Collection

Approximately 20 ml blood was obtained via venipuncture after informed consent from patient and Next of Kin or Legal Guardian. Genomic DNA was prepared from blood by using the Gentra whole blood kit according to the manufacturer's instructions.

AAO Phenotyping

AAO is determined by two standardized methods at all 4 contributing sites: i) unprompted caregiver estimate regarding onset of symptoms and ii) physician estimate of duration of illness using a standardized and validated structured interview with landmark event to facilitate recall (Doody et al. 2004). The procedure entails a questionnaire with 34 questions asking the onset of symptoms affecting domains that may be associated with AD: memory, language, orientation and executive function; and behavioral-psychiatric symptoms. Subsequently the AAO was confirmed by assessing functional status of the patient before, at and after a major lifetime event dating to the estimated AAO (use of landmark event to assist recall).

The retrospective ascertainment of AAO may result in inaccuracies in recall. While prospective phenotyping of age at onset of memory problems (delayed recall) or functional decline (CDR) would be ideal, prospective cohort studies (Framingham, ACT) have a conversion rate of 0.6-1% per year, thus this is not feasible for this study. Two retrospective methods are used which are standardized between sites and the Pearson correlation is significant (p<0.0001) between the two methods. It is encouraging, that the APOE4 effect on age at onset was found in multiple previous studies with retrospective phenotyping of AAO as well.

Clinical and Neuropsychological Data

In addition to age at onset of symptoms (if AD patient), information obtained from the clinical and neurological examination on family history of dementia in first degree relatives, cardiovascular disease and cardiovascular disease risk factors, education, history of head trauma and smoking were gathered on each subject. The following neuropsychological tests administered as part of the routine dementia work-up at each site were entered into the database: Global cognitive functioning/status (Mini Mental Status Exam (MMSE) and Clinical Dementia Rating (CDR)); Attention (Digit Span and Trails A); Executive function (Trails B and Clock Drawing; Texas Card Sorting is optional); Memory (Wechsler Memory Scale (WMS) Logical Memory I and WMS Logical Memory II); Language (Boston Naming and FAS Verbal Fluency); Premorbid IQ (American National Adult Reading Test (AMNART); Visuospatial Memory (WMS-Visual Reproduction I and II); Psychiatric (Geriatric Depression Scale; Neuropsychiatric Inventory-Questionnaire); Functional (Lawton-Brody Activities of Daily Living (ADL): Physical Self Maintenance Scale, Instrumental Activities of Daily Living (IADL))

Enrollment Status: 521 Cases and 228 Controls

TABLE 7
Demographic characteristics of the cohort.

| | Cases (N = 521) | Controls (N = 228) |
|---|---|---|
| Sex Female/male (%) | 58.9/41.2 | 64/36 |
| Mean age (SD) | 76.7 (8.3) | 71.1 (8.6) |
| Mean age at onset (SD) | 71.1 (8.6) | NA |
| Race | | |
| Caucasian (%) | 469 (90.2%) | 212 (93%) |
| African-American (%) | 26 (5%) | 4 (1.8%) |
| Hispanic | 16 (3.1%) | 4 (1.8%) |
| Other | 9 (1,.7%) | 8 (3.5%) |
| Education Mean (SD) | 14.3 (3.2) | 15.4 (2.8) |
| MMSE Mean (SD) | 20 (5.7) | 29.4 (0.9) |
| CDR Sum Mean (SD) | 6.9 (3.9) | 0.03 (0.13) |

### Example 8

A custom aCGH is designed covering all OR genes to comprehensively study the ORs.

Custom Tiling aCGH

A tiling oligo CGH array is developed to achieve complete coverage of the OR regions. The 8×15 k array design may be used for feasibility. The OR regions cover approximately 72 Mb genomic sequence, the 15,000 probes will provide an approximately 1 probe/5 kb density. It is aimed to cover the OR genes and pseudogenes (0.7 Mb) with a density of at least 4 probes per exon and the remaining probe allowance will be distributed along the OR regions in non-repeat sequences and utilized for padding on both sides of the OR region to create a backbone (itraindividual diploid reference). A 50% overlap between probes (60-mers) is allowed for.

Array Design

The OR genomic coordinates are obtained from two published datasets and these will be mapped to the Build 36 assembly of the human genome. The regions will be manually confirmed in the UCSC genome browser. eArray is used to select probes from the Agilent library to cover the OR regions. Selection criteria will be the following: Empirically and bioinformatically tested probes over bioinformatically only tested probes; Bioinformatically tested probes over not tested probes; For regions where adequate probe density is not achieved from library probes are designed by manually searching for unique sequences.

### Example 9

Custom aCGH is performed on the TARC cohort to assess the effect size of all ORs.

All ORs are aimed to be characterized including those that are not covered on the commercially available arrays but have a comparable or larger effect size to the OR-CNV identified in the above examples. Normalized numeric hybridization data and CNV parallel calls are used as they will result in overlapping but distinct lists of the most significant association. Analysis of the raw normalized hybridization data might identify small segments that the CNV calling algorithm would have ignored. Two distinct statistical methods are proposed, hazard function regression and sequential addition of cases to identify CNVs that are associated with AAO variation across the entire AAO distribution and CNVs that have stronger effects in subsets of families defined by a continuous covariate. Analysis may not necessarily be limited to polymorphic CNVs published in databases; rather all copy number changes may be ascertained. In embodiments of the invention i) a subset of genes contributing to the heritability of AD do not cause the disease by themselves but, in combination with other genes or epigenetic factors, modulate the AAO and increase the probability of developing AD in the individual's lifespan, ii) if modified, it may result in a significant reduction of public health burden (5 year delay will reduce it by half by the year 2047, iii) it is determined by the evaluating physician by standardized and validated procedures in the TARC protocol, and/or iv) the proof of principle that AAO analysis is powerful in identifying risk factors for AD was shown for APOE in a cohort of 40 patients by sequential addition of cases.

The aCGH developed in Example 8 is performed on 600 subjects (500 AD and 100 control) of the same cohort to assess the effect size of all OR regions. The TARC cohort will be the replication study for the chromosome 14q11.2 region using the subset of subjects that have not been studied in the previous 2 cohorts described in the preliminary data (N=286) and the discovery cohort for all other OR regions (FIG. 4).

Identification of regions where CN state is significantly associated with AAO within the case population is focused on by hazard regression analysis using the normalized quantitative array data and parallel the called CNVs. Sequential addition method is used to examine the statistical association of the normalized quantitative CN information and parallel the called CNVs with AAO in ordered subsets of cases by testing against the controls. Those CNVs which (a) are significantly associated with AAO in the case population or (b) which are significantly different between cases and controls according to the sequential addition analysis will be the candidate CNVs for future replication. The multiple testing problem is handled by employing an FDR approach regression step (Keles et al. 2006). The four parallel analysis methods is corrected for. Resampling methods are utilized to improve our initial p-value estimation in both the regression and the sequential addition steps.

Also included are i) perform quality control steps, ii) assess population substructure and admixture by the available SNP dataset iii) perform AAO analysis iv) and perform the SA association analysis.

Power analysis for the AAO study was performed by directly resampling the copy number array data and sample information for the data cohort described above (see first 7 examples) using the region identified on chromosome 14 as a target. Because this region may be unusual in terms of both the size of the effect and strong allelic variation, additional methods are constructed to both reduce the effect size and limit the allelic variation in order to make the power calculation more complete. The estimates were produced by performing the hazard regression analysis on 100 instances of resampled data for each sample size, effect size (median difference in AAO) and allelic composition. For each run the percentage of times the geometric mean of the regression outcome −log p-values surpassed the threshold of 7 were counted and were reported as the power of the study.

To implement the comparison for smaller effect size, the AAO values were randomly shifted by 5 years for random samples of half the patients from the high copy number cohort and half the patients from the low copy number class, where the classes are defined by median log-ratio value in the preliminary data cohort. This adjustment effectively lowers the AAO effect size to an average of 5 years difference in median AAO between classes from its initial value of approximately 10 years. The power to detect the copy number effect on AAO for sample sizes between 50 and 500 are depicted in table 8.

TABLE 8
Calculated power to detect the copy number effect on
AAO difference of 10 and 5 years for sample sizes of
50-500 for a region of similar allele frequency as the
chromosome 14 region in the preliminary studies.

| Median | | | | | | | | | |
|---|---|---|---|---|---|---|---|---|---|
| AAO | Sample size | | | | | | | | |
| difference | 0 | 00 | 50 | 00 | 50 | 00 | 50 | 00 | 00 |
| 0 years | 9% | 9% | 00% | 00% | 00% | 00% | 00% | 00% | 00% |
| years | % | 3% | 8% | 1% | 3% | 9% | 1% | 7% | 9% |

Additional simulations were performed to reduce the amount of allelic variation by approximately 40% (Table 9). This analysis was accomplished by adding a component to the simulation where half the individuals of each copy number class (gain or loss) were randomly sampled from the initial cohort and replaced their data values with a random sample of Gaussian noise with mean 0 and variance determined as half the IQR of the ensemble of log-ratio values. This removal of allelic variation corresponds to copy number variation appearing in approximately 18% of samples as opposed to 30% for the original data cohort. Power calculations in the context of reduced allelic variation for median age at onset differences of both 10 and 5 years appear in the table below, where the reduction in effect size (median difference in AAO) is accomplished independently from the reduction in allelic variation according to the method described above.

TABLE 9
Calculated power to detect the copy number effect on AAO difference
of 10 and 5 years for sample sizes of 50-500 for a region
where the CNV is detected in 18% of the population.

| Median | | | | | | | | | |
|---|---|---|---|---|---|---|---|---|---|
| AO | Sample size | | | | | | | | |
| difference | 0 | 00 | 50 | 00 | 50 | 00 | 50 | 00 | 00 |
| 0 years | 1% | 3% | 4% | 7% | 1% | 5% | 5% | 6% | 00% |
| years | % | 0% | 1% | 8% | 3% | 9% | 3% | 7% | 4% |

The power analysis suggests that a sample size of 500 is able to detect AAO effects when they are present in the population if the CNV is common or the effect is large.

Power analysis for the sequential addition approach was performed using the data from Examples 1-7 to provide distribution estimates for the CNV data. A simulation procedure was constructed to evaluate the ability to detect differences between AD patients and controls by drawing with replacement from the empirical distribution of our pilot data. A sample size of 100 controls and sample sizes of 50, 100, 200, 300, 400, and 500 of AD patients was evaluated. The power to detect differences is approximately 75% for a sample size of 50 AD patients. As the sample size increases, a steady increase in power is observed reaching over 90% for an AD sample size of >500. The numbers are consistent with the high power of the sequential approach in the APOE study (Macgregor et al. 2006).

Agilent 15k aCGH

The procedures will be performed according to the manufacturer's instructions. Briefly, for each aCGH hybridization, 0.5 μg of genomic DNA is digested from the reference subject and the experimental sample with AluI (20 units) and RsaI (20 units) (Promega). Labeling reactions will be performed with 6 μg of purified restricted DNA and a Genomic DNA labeling kit (Agilent) according to the manufacturer's instructions with hCy5-dUTP (for the experimental sample) or Cy3-dUTP (for the reference) (PerkinElmer). After denaturation the sample will be applied to the array by using an Agilent microarray hybridization chamber, and hybridization will be carried out for 40 h at 65° C. in a rotating oven (Robbins Scientific, Mountain View, Calif.) at 4 rpm. The arrays are then disassembled and washed slides are dried and scanned by using an Agilent 2565AA DNA microarray scanner. For quality control the average signal strengths are analyzed across the arrays and derivative log ratio spreads (dlrs). Dlrs may be less than 0.3 to be included in the study.

Experimental Quality Control

Per array QC algorithms will be performed as standard protocol available in the manufacturer's analysis package. Derivative log ratio spread (dlrs)<0.4 may be required for CNV analysis.

Data Quality Control

Arrays may be removed that have numbers of CNV calls more than two SD from the mean. Currant algorithms cannot compensate for uneven baseline and generate an excessive number of calls. These arrays may be excluded from the hazard function regression and sequential addition of cases analyses due to the high false positive call rate for that specific array.

Estimating Marker Specific Error Rates

In order to assess marker-specific error rates technical duplicates may be performed on 2% of the samples.

Population Substructure/Admixture by the SNP Dataset

Non-Caucasian samples may be excluded. TARC SNP data available is utilized to estimate population substructure in the Caucasian cohort and utilize this data in the CN analysis. Several statistically valid methods for estimating and correcting such structure have been developed, including the structured association (SA) and principal components methods (Devlin and Roeder 1999; Pritchard et al. 2000; Hoggart et al. 2003; Patterson et al. 2006; Price et al. 2006). For multi-locus SNP data, SA assigns the samples to discrete subpopulation clusters and then tests for association within each cluster. Because cluster membership is specified in a K-length vector estimated using MCMC-based inference, the method is very computationally intensive. A practical alternative is provided by the principal components (PC) method (Patterson et al. 2006; Price et al. 2006). PC is applied to the full SNP genotype data to infer continuous axes of genetic variation. This produces the expected dimension reduction that maximizes the explained variability (first few eigenvectors of the between-sample covariance matrix). Linear regressions are used to continuously adjust genotypes by the amounts attributable to ancestry along each axis. Association statistics are computed using ancestry-adjusted genotypes and phenotypes. These methods are incorporated into the EIGENSOFT package.

Numerical Array Data

The analysis program in R has been created (see above examples) to perform AAO regression with the numerical CNV array data. This approach searches for genomically contiguous regions where CN state has a strong AO effect. The strong AAO effect is computationally determined using a maximum likelihood analysis that treats the AAO times as random variables dependent on the CN state (hazard regression). To enhance the analysis a “thin and bin” may be used to approach the hazard analysis.

Thinning and Binning

Every other oligo represented on the array is sampled to divide the data in half. In each half, K genomically adjacent oligos are bined and regression of AAO is run on the mean CNV state within each thinned bin for each patient. FDR values are computed for each thin bin p value, and rank the q-values for the CNV's coefficient from lowest (near 0) to highest (near 1) in each half. In the initial work K=10 was used, but other choices of K are possible, such as choices of K between K=2 and K=100. It is not clear which value of K will yield optimal results, but the thinning approach gives a direct method to identify the K at which maximum concordance is attained between highest scoring significant loci identified in the 2 halves. K is identified at which maximum concordance is attained with FDR q values less than 0.05 in each data half. The direction of effect (sign of the beta coefficient) is verified, and these common high scoring CNVs (low FDR q-values) are designated in the two halves as CNV set N1.

Effects of Moderate Size

The regression of age at onset is run on the CNVs on the entire dataset removing the thinning but retaining data aggregation into K oligo bins. The FDR q-values are ranked and CNVs not belonging to set N1 but with significant q-values (less than 0.05) are selected and these CNV set are called N2.

Called CNVs

Sliding-window algorithm in the Agilent software package are used to infer CN state. The hazard function regression is performed on AAO using the CNV dosage as covariate. The FDR q-values are ranked and CNVs not belonging to set N1 and N2 but with significant q-values (less than 0.05) are selected and these CNV are called set N3.

Association Analysis by Sequential Addition of Cases

To identify loci affecting AAO against the control data sequential addition analysis is performed as described in Macgregor et al. (Macgregor et al. 2006) Briefly, for each CNV, AAO is sorted from youngest to oldest and then oldest to youngest. The AD patients are compared with controls step-wisely. The CNV scores of the first k nested subsets of age-ranked AD patients are analyzed with the controls using both a parametric (t-test) and non-parametric (wilcoxon test) comparison of means for the CNV data (numeric and called). The p-values are adjusted for the multiple CNVs using the FDR approach. The FDR q-values are ranked and CNVs not belonging to set N1, N2 and N3 but with significant q-values (less than 0.05) are selected and called set N4.

Weights to regression analysis may be added or random covariate analysis may be performed to adjust for uncertainty in AAO. Alternatively ordinal analysis may be applied where AAO is binned into either 2 or a small number of classes so that the data is not used numerically. Since the number of such commonly variant intervals is moderate compared with the size of the array, multiple testing is less of a challenge and will be handled using FDR methods. This analysis also avoids arbitrarily binning the data and instead defines intervals to be tested in a data-dependent fashion that still incorporates the AAO analysis.

### Example 10

Olfactory deficits in normal aging, cognitive decline, mild cognitive impairment and AD have been described in clinical studies. Cognitive profiles associated with olfactory deficit have been studied in older adults and AD. In older adults olfactory identification was associated with impairment of memory and language in one study. Cognitive deficits of subjects with AD with and without olfactory deficit have been reported to affect visuospatial function; however, that example may have not been powered to detect the memory and language differences, which would be expected from the anatomical pathway of smell.

Odour identification will be measured by the UPSIT measure at the time of the annual follow-up visit at each site after reconsenting the subject with the amended consent form, which includes olfactory testing. The reliability of the test is not established in subjects with MMSE less than 14; subjects in this part of the study are included who have an MMSE over 14. The UPSIT test will be administered at the time of the annual follow-up visit. The neuropsychological battery at presentation is assessed as part of the standard clinical evaluation and is available from the TARC database.

### Primary Outcome Measure

The UPSIT is used to measure olfactory identification. The UPSIT is a multiple-forced-choice odor identification test. For each odorant there are four possible responses and the subject is required to choose one even of smell is not perceived. It requires 10-15 minutes to administer. The UPSIT consists of 40 odorants in 4 booklets, 10 odorant in each booklet. The odorant is located on a brown strip in microencapsulated crystals at suprathreshold level. The strip has to be scratched with a pencil and then one of the 4 choices marked. The measure has been validated for short-term, long-term and test-retest reliability. (Doty et al. 1989) Normative data for the UPSIT include a score on the 1-40 scale and percentile ranks for men and women across the entire age span. The UPSIT measures odor identification and discrimination. The complete 40 item set is used to ascertain potential correlation of the specific genotype with loss of specific odor recognition.

### Secondary Outcome Measures

Neuropsychological measures ascertained at the time of enrollment to TARC will be compared between the various copy number sates of the regions showing association with age at onset of AD to identify the cognitive endophenotype associated with the olfactory genotype if present. Previous exploratory studies have found differences in memory, language and visuospatial function in subjects with or without clinical olfactory deficit. Memory deficits can be explained by the anatomical connection of the olfactory pathway to the medial temporal lobe structures and thus olfactory deficit may be an early clinical manifestation. The approach using the olfactory genotypes may reduce the noise affecting analyses based on clinical phenotypes, thus prove more powerful as previous studies. The copy number genotype may be associated with cognitive endophenotypes irrespective of clinical olfactory deficit, as the OR genes are expressed in other brain areas and may have functions outside of the olfactory pathway. The following domains are assessed and analysis is completed for the domains studied, not limiting to the previously found associations with clinical deficit: Global cognitive functioning/status (MMSE and CDR); Attention (Digit Span and Trails A); Executive function (Trails B and Clock Drawing; Texas Card Sorting is optional); Memory (WMS Logical Memory I and WMS Logical Memory II); Language (Boston Naming and FAS Verbal Fluency); Premorbid IQ (AMNART); Visuospatial Memory (WMS-Visual Reproduction I and II); Psychiatric (Geriatric Depression Scale; Neuropsychiatric Inventory-Questionnaire); and Functional (Lawton-Brody ADL: PSMS, IADL)

Statistical Analysis:

The association of the CNV states with the UPSIT global score and individual odorant items will be assessed by a series of regression models that control for the potentially confounding effects of age, sex, education and smoking. The cognitive measures between groups defined by the OR CN states will be compared by parametric (t-test, analysis of covariance) and nonparametric (Wilcoxon test) statistics, as appropriate. Corrections for multiple comparisons will be applied using both False Discovery Rate methodology as well as analysis of Family Wise Error Rate based on permutation computed p-values.

### Example 11

The expression analysis of OR genes in human brain tissue is expanded to test the hypothesis of dysregulation of OR expression. In one embodiment of the invention, altered OR copy number may perturb the allelic exclusion characteristic of OR receptor expression. Allelic expression from an allele harboring two copies of the sequence may mimic biallelic expression. This can be studied by sequence analysis of the RT-PCR transcripts using the SNPs within the sequence to determine which copy of which allele is expressed.

RNA and DNA was isolated from temporal lobes of post mortem brain specimens from subjects with AD and age-matched controls. 14 samples were obtained from the BCM/Methodist Hospital tissue repository and 28 samples from the New York Brain Bank (NYBB). The samples were deidentified. Illumina expression array and Affymetrix 6.0 array were performed on each sample as described in the preliminary data. Array level expression was detected for a subset of ORs. It is proposed to perform the aCGH from Example 8 to determine CN state for the brain specimens. The number 42 will sample CNVs that are present in 2% of the population. RT-qPCR assays is performed for transcripts of the OR genes that showed association with AAO of AD (candidate OR) in Example 9. The olfactory bulb is used in addition to the temporal lobe to study expression of the candidate ORs.

Specimens

The temporal lobe DNA and RNA are already extracted as presented in the previous examples. Ten subjects had available olfactory bulb specimens which were obtained from the NYBB. Corresponding brain tissue was examined by a neuropathologist and confirmed the diagnosis of definite AD. 1 g of olfactory bulb will be removed for RNA isolation. For RNA preparation, brain tissue is homogenized with a tissue homogenizer in Trizol (Invitrogen) followed standard procedures and further purified with Rneasy mini kit (Qiagen). Quality control measures for RNA include absorption at 260/280 nm of 1.9-2.0, at 260/230 nm>1.5 by Nanodrop 1000 and visual inspection of agarose gels

Agilent 15k aCGH

The aCGH developed in Example 8 is performed on the brain cohort. The procedures will be performed according to the manufacturer's instructions, as described in Example 9. For quality control the average signal strengths are analyzed across the arrays and derivative log ratio spreads (dlrs). The dlrs may be less than 0.3 to be included in the study.

Perturbation of OR Expression

RT-PCR expression analysis is performed on the RNA from the temporal lobes and olfactory bulbs. If the candidate OR RNA is detected, RT-PCR is performed on single cells by laser capturing neurons. The single cell experiments will allow assessing whether the duplication results in expression of both copies and thus may mimic biallelic expression. RT-PCR the mRNA of OR genes identified in Example 9 is done and the fragments are subject to sequencing. The SNPs differing between copy 1 and copy 2 on one allele and the copy on the second allele will suggest whether the allelic exclusion is perturbed by the CN state.

### Example 12

### Exemplary Clinical Embodiment of the Invention

In certain embodiments of the invention, an individual suspected of having or being at risk for early onset of AD provides a sample (which may comprise cells) that includes genomic DNA. The copy number of the genomic DNA is determined for at least part of the region at chromosome 14q11.2, which may include, for example, reference sequence position 19.3-19.5 Mb and may include at least part of one or more of an olfactory receptor gene, including, for example, one or more of OR4M1, OR4N2, OR4K2, OR4K5, or OR4K1. When the copy number is high for the individual, the individual is at greater risk of having early AAO (for example, onset of AAO being at less than 65 years of age). In certain cases, when an individual has, will have, or is at greater risk of having early AAO, the individual may be provided one or more therapies to treat AD or at least treat one or more of its symptoms, including cognitive function and/or behavioral symptoms. The CN may also be determined for olfactory receptor genes.

### REFERENCES

All patents and publications mentioned in the specification are indicative of the levels of those skilled in the art to which the invention pertains. All patents and publications are herein incorporated by reference to the same extent as if each individual publication was specifically and individually indicated to be incorporated by reference.
Bertram L, Lange C, Mullin K, et al. Genome-wide association analysis reveals putative Alzheimer's disease susceptibility loci in addition to APOE. Am J Hum Genet 2008; 83:623-32.
Bertram L, Tanzi RE. Thirty years of Alzheimer's disease genetics: the implications of systematic meta-analyses. Nat Rev Neurosci 2008; 9:768-78.
Brookmeyer R, Gray S, Kawas C. Projections of Alzheimer's disease in the United States and the public health impact of delaying disease onset. Am J Public Health 1998; 88:1337-42.
Carter, N. P. (2007). “Methods and strategies for analyzing copy number variation using DNA microarrays.” Nat Genet 39(7 Suppl): S 16-21.
Chess A, Simon I, Cedar H, Axel R. Allelic inactivation regulates olfactory receptor gene expression. Cell 1994; 78:823-34.
Cooper, G. M., T. Zerr, et al. (2008). “Systematic assessment of copy number variant detection via genome-wide SNP genotyping.” Nat Genet 40(10): 1199-203.
Conrad D F, Hurles M E. The population genetics of structural variation. Nat Genet 2007; 39:S30-6.
Corder E H, Saunders A M, Strittmatter W J, et al. Gene dose of apolipoprotein E type 4 allele and the risk of Alzheimer's disease in late onset families. Science 1993; 261:921-3.
Devanand, D. P., K. S. Michaels-Marston, et al. (2000). “Olfactory deficits in patients with mild cognitive impairment predict Alzheimer's disease at follow-up.” Am J Psychiatry 157(9): 1399-405.
Devlin, B. and K. Roeder (1999). “Genomic control for association studies.” Biometrics 55(4): 997-1004.
Djordjevic, J., M. Jones-Gotman, et al. (2008). “Olfaction in patients with mild cognitive Doody R, Pavlik V, Massman P, et al. Changing patient characteristics and survival experience in an Alzheimer's center patient cohort. Dement Geriatr Cogn Disord 2005; 20:198-208.
Doody R S, Dunn J K, Huang E, Azher S, Kataki M. A method for estimating duration of illness in Alzheimer's disease. Dement Geriatr Cogn Disord 2004; 17:1-4. impairment and Alzheimer's disease.” Neurobiol Aging 29(5): 693-706.
Doody, R., V. Pavlik, et al. (2005). “Changing patient characteristics and survival experience in an Alzheimer's center patient cohort.” Dement Geriatr Cogn Disord 20(2-3): 198-208.
Doty, R. L., R. E. Frye, et al. (1989). “Internal consistency reliability of the fractionated and whole University of Pennsylvania Smell Identification Test.” Percept Psychophys 45(5): 381-4.
Ebly, E. M., I. M. Parhad, et al. (1994). “Prevalence and types of dementia in the very old: results from the Canadian Study of Health and Aging.” Neurology 44(9): 1593-600.
Eibenstein, A., A. B. Fioretti, et al. (2005). “Modern psychophysical tests to assess olfactory function.” Neurol Sci 26(3): 147-55.
Fanciulli, M., P. J. Norsworthy, et al. (2007). “FCGR3B copy number variation is associated with susceptibility to systemic, but not organ-specific, autoimmunity.” Nat Genet 39(6): 721-3.
Feldmesser, E., T. Olender, et al. (2006). “Widespread ectopic expression of olfactory receptor genes.” BMC Genomics 7: 121.
Feuk, L., A. R. Carson, et al. (2006). “Structural variation in the human genome.” Nat Rev Genet 7(2): 85-97.
Graves A B, Bowen J D, Rajaram L, et al. Impaired olfaction as a marker for cognitive decline: interaction with apolipoprotein E epsilon4 status. Neurology 1999; 53:1480-7.
Gatz, M., C. A. Reynolds, et al. (2006). “Role of genes and environments for explaining Alzheimer disease.” Arch Gen Psychiatry 63(2): 168-74.
Gonzalez, E., H. Kulkarni, et al. (2005). “The influence of CCL3L1 gene-containing segmental duplications on HIV-1/AIDS susceptibility.” Science 307(5714): 1434-40.
Graves, A. B., J. D. Bowen, et al. (1999). “Impaired olfaction as a marker for cognitive decline: interaction with apolipoprotein E epsilon4 status.” Neurology 53(7): 1480-7.
Hoggart, C. J., E. J. Parra, et al. (2003). “Control of confounding of genetic associations in stratified populations.” Am J Hum Genet 72(6): 1492-1504.
Hoogendijk, J. E., G. W. Hensels, et al. (1992). “De-novo mutation in hereditary motor and sensory neuropathy type I.” Lancet 339(8801): 1081-2.
Joachim, C. L., J. H. Morris, et al. (1988). “Clinically diagnosed Alzheimer's disease: autopsy results in 150 cases.” Ann Neurol 24(1): 50-6.
Keles, S., M. J. van der Laan, et al. (2006). “Multiple testing methods for ChIP-Chip high density oligonucleotide array data.” J Comput Biol 13(3): 579-613.
Kukull, W. A., R. Higdon, et al. (2002). “Dementia and Alzheimer disease incidence: a prospective cohort study.” Arch Neurol 59(11): 1737-46.
Kukull, W. A., C. Martin, et al. (2002). “Dementia and Alzheimer disease incidence: a prospective cohort study.” Arch Neurol 59(3): 664-70.
Kurtz, P., T. Schuurman, et al. (1989). “Loss of smell leads to dementia in mice: is Alzheimer's disease a degenerative disorder of the olfactory system?” J Protein Chem 8(3): 448-51.
Lachman, H. M., E. Pedrosa, et al. (2007). “Increase in GSK3beta gene copy number variation in bipolar disorder.” Am J Med Genet B Neuropsychiatr Genet 144(3): 259-65.
Li, H. and J. Fan (2000). “A general test of association for complex diseases with variable age of onset.” Genet Epidemiol 19 Suppl 1: S43-9.
Lupski JR. Structural variation in the human genome. N Engl J Med 2007; 356:1169-71.
Macgregor, S., N. Craddock, et al. (2006). “Use of phenotypic covariates in association analysis by sequential addition of cases.” Eur J Hum Genet 14(5): 529-34.
McCarroll SA. Extending genome-wide association studies to copy-number variation. Hum Mol Genet 2008; 17:R135-42.
McCarroll, S. A. and D. M. Altshuler (2007). “Copy-number variation and association studies of human disease.” Nat Genet 39(7 Suppl): S37-42.
McKhann, G., D. Drachman, et al. (1984). “Clinical diagnosis of Alzheimer's disease: report of the NINCDS-ADRDA Work Group under the auspices of Department of Health and Human Services Task Force on Alzheimer's Disease.” Neurology 34(7): 939-44.
Mesholam, R. I., P. J. Moberg, et al. (1998). “Olfaction in neurodegenerative disease: a meta-analysis of olfactory functioning in Alzheimer's and Parkinson's diseases.” Arch Neurol 55(1): 84-90.
Nakajima T, Kaur G, Mehra N, Kimura A. HIV-1/AIDS susceptibility and copy number variation in CCL3L1, a gene encoding a natural ligand for HIV-1 co-receptor CCR5. Cytogenet Genome Res 2008; 123:156-60.
Nelis, E., C. Van Broeckhoven, et al. (1996). “Estimation of the mutation frequencies in Charcot-Marie-Tooth disease type 1 and hereditary neuropathy with liability to pressure palsies: a European collaborative study.” Eur J Hum Genet 4(1): 25-33.
Nozawa, M., Y. Kawahara, et al. (2007). “Genomic drift and copy number variation of sensory receptor genes in humans.” Proc Natl Acad Sci USA 104(51): 20421-6.
Ohm T G, Braak H. Olfactory bulb changes in Alzheimer's disease. Acta Neuropathol 1987; 73:365-9.
Padiath, Q. S., K. Saigoh, et al. (2006). “Lamin B1 duplications cause autosomal dominant leukodystrophy.” Nat Genet 38(10): 1114-23.
Patterson, N., A. L. Price, et al. (2006). “Population structure and eigenanalysis.” PLoS Genet 2(12): e190.
Price, A. L., N. J. Patterson, et al. (2006). “Principal components analysis corrects for stratification in genome-wide association studies.” Nat Genet 38(8): 904-9.
Pritchard, J. K., M. Stephens, et al. (2000). “Inference of population structure using multilocus genotype data.” Genetics 155(2): 945-59.
Raiha I, Kaprio J, Koskenvuo M, Rajala T, Sourander L. Alzheimer's disease in Finnish twins. Lancet 1996; 347:573-8.
Redon, R., S. Ishikawa, et al. (2006). “Global variation in copy number in the human genome.” Nature 444(7118): 444-54.
Rice, D. P., P. J. Fox, et al. (1993). “The economic burden of Alzheimer's disease care.”Health Aff (Millwood) 12(2): 164-76.
Rocca, W. A., A. Hofman, et al. (1991). “Frequency and distribution of Alzheimer's disease in Europe: a collaborative study of 1980-1990 prevalence findings. The EURODEM-Prevalence Research Group.” Ann Neurol 30(3): 381-90.
Rovelet-Lecrux, A., D. Hannequin, et al. (2006). “APP locus duplication causes autosomal dominant early-onset Alzheimer disease with cerebral amyloid angiopathy.” Nat Genet 38(1): 24-6.
Scherer, S. W., C. Lee, et al. (2007). “Challenges and standards in integrating surveys of structural variation.” Nat Genet 39(7 Suppl): S7-15.
Schouten J P, McElgunn C J, Waaijer R, Zwijnenburg D, Diepvens F, Pals G. Relative quantification of 40 nucleic acid sequences by multiplex ligation-dependent probe amplification. Nucleic Acids Res 2002; 30:e57.
Schubert, C. R., L. L. Carmichael, et al. (2008). “Olfaction and the 5-year incidence of cognitive impairment in an epidemiological study of older adults.” J Am Geriatr Soc 56(8): 1517-21.
Scott, W. K., E. R. Hauser, et al. (2003). “Ordered-subsets linkage analysis detects novel Alzheimer disease loci on chromosomes 2q34 and 15q22.” Am J Hum Genet 73(5): 1041-51.
Sereda M W, Meyer z u Horste G, Suter U, Uzma N, Nave K A. Therapeutic administration of progesterone antagonist in a model of Charcot-Marie-Tooth disease (CMT-1A). Nat Med 2003; 9:1533-7
Singleton, A. B., M. Farrer, et al. (2003). “alpha-Synuclein locus triplication causes Parkinson's disease.” Science 302(5646): 841.
Shykind B M, Rohani S C, O'Donnell S, et al. Gene switching and the stability of odorant receptor gene choice. Cell 2004; 117:801-15.
Stankiewicz P, Thiele H, Giannakudis I, et al. Kabuki syndrome-like features associated with a small ring chromosome X and XIST gene expression. Am J Med Genet 2001; 102:286-92.
Stranger, B. E., M. S. Forrest, et al. (2007). “Relative impact of nucleotide and copy number variation on gene expression phenotypes.” Science 315(5813): 848-53.
Stefansson H, Rujescu D, Cichon S, et al. Large recurrent microdeletions associated with schizophrenia. Nature 2008; 455:232-6.
Waring S, O'Bryant S, Reisch J, Diaz-Arrastia R, Knebl J, Doody R. The Texas Alzheimer's Research Consortium longitudinal research cohort: Study design and baseline characteristics. Texas Public Health Journal 2008; 60:9-13.
Willcocks L C, Lyons P A, Clatworthy M R, et al. Copy number of FCGR3B, which is associated with systemic lupus erythematosus, correlates with protein expression and immune complex uptake. J Exp Med 2008; 205:1573-82.
Yang, Y., E. K. Chung, et al. (2007). “Gene copy-number variation and associated polymorphisms of complement component C4 in human systemic lupus erythematosus (SLE): low copy number is a risk factor for and high copy number is a protective factor against SLE susceptibility in European Americans.” Am J Hum Genet 80(6): 1037-54.
Young J M, Endicott R M, Parghi S S, Walker M, Kidd J M, Trask B J. Extensive copy-number variation of the human olfactory receptor gene family. Am J Hum Genet 2008; 83:228-42.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

What is claimed is:

1. A method for assaying for a risk factor for early age at onset (AAO) for Alzheimer's Disease in an individual, comprising the step of assaying copy number variation (CNV) on chromosome 14q11.2 from a sample from the individual.

2. The method of claim 1, wherein the copy number variation occurs within a gene locus comprising one or more members of the olfactory receptor gene cluster.

3. The method of claim 2, wherein the copy number variation corresponds to one or more genes selected from the group consisting of OR4M1, OR4N2, OR4K2, OR4K5, and OR4K1.

4. The method of claim 1, wherein when there is an increase in copy number within one or more loci in chromosome 14q11.2, the individual will have a higher risk for an earlier AAO.

5. The method of claim 4, wherein when the individual will have an earlier AAO, the individual is provided therapy for Alzheimer's Disease.

6. The method of claim 1, wherein the risk factor indicates that the individual's AAO will be before age 65, 60 or 55.

7. The method of claim 1, wherein the assay comprises multiplex ligation-dependent probe amplification.

8. The method of claim 1, wherein a higher risk is assessed if the individual also has APOE4/4.

9. The method of claim 1, wherein a higher risk is assessed if the CNV is 3, 4, 5, or greater than 5.

10. The method of claim 9, wherein a higher risk is assessed if the CNV is 4, 5, or higher than 5.

11. The method of claim 10, wherein a higher risk is assessed if the CNV is 5, or higher than 5.

12. A method for assaying for a risk factor for early age at onset (AAO) for Alzheimer's Disease in an individual, comprising the step of assaying copy number variation (CNV) on an olfactory receptor gene from the individual.
